SEQUENCE LISTING (12 sequences)

SEQ ID NO: 1 - DNA, 20 bp, synthetic construct
  misc_feature 1..20: note = Synthetic oligonucleotide
  source 1..20: mol_type = other DNA
atcacacccgtcacacatcc

SEQ ID NO: 2 - DNA, 20 bp, synthetic construct
  misc_feature 1..20: note = Synthetic oligonucleotide
  source 1..20: mol_type = other DNA
ggggtgatctgtccaacacc

SEQ ID NO: 3 - DNA, 20 bp, synthetic construct
  misc_feature 1..20: note = Synthetic oligonucleotide
  source 1..20: mol_type = other DNA
tctgctctgaagtcaacggc

SEQ ID NO: 4 - DNA, 20 bp, synthetic construct
  misc_feature 1..20: note = Synthetic oligonucleotide
  source 1..20: mol_type = other DNA
gcctcacagtgctcgatctt

SEQ ID NO: 5 - DNA, 20 bp, synthetic construct
  misc_feature 1..20: note = Synthetic oligonucleotide
  source 1..20: mol_type = other DNA
cgcgaagacaggcatcaaag

SEQ ID NO: 6 - DNA, 20 bp, synthetic construct
  misc_feature 1..20: note = Synthetic oligonucleotide
  source 1..20: mol_type = other DNA
cgtatgtgaaggccgtctcg

SEQ ID NO: 7 - DNA, 19 bp, synthetic construct
  misc_feature 1..19: note = Synthetic oligonucleotide
  source 1..19: mol_type = other DNA
tgcgctgaaccttcgactg

SEQ ID NO: 8 - DNA, 22 bp, synthetic construct
  misc_feature 1..22: note = Synthetic oligonucleotide
  source 1..22: mol_type = other DNA
agcgtggaaattgtctgttctg

SEQ ID NO: 9 - DNA, 20 bp, synthetic construct
  misc_feature 1..20: note = Synthetic oligonucleotide
  source 1..20: mol_type = other DNA
aggacaagatggagctgctg

SEQ ID NO: 10 - DNA, 20 bp, synthetic construct
  misc_feature 1..20: note = Synthetic oligonucleotide
  source 1..20: mol_type = other DNA
atctttcccagtgtcgccag

SEQ ID NO: 11 - DNA, 20 bp, synthetic construct
  misc_feature 1..20: note = Synthetic oligonucleotide
  source 1..20: mol_type = other DNA
gggcacggctataaaccact

SEQ ID NO: 12 - DNA, 20 bp, synthetic construct
  misc_feature 1..20: note = Synthetic oligonucleotide
  source 1..20: mol_type = other DNA
tccttctgccaaccttccac

(19) United States
(12) Patent Application Publication
(10) Pub. No.: US 2024/0279609 A1
(43) Pub. Date: Aug. 22, 2024

(54) METHOD FOR ISOLATING HUMAN DERMAL FIBROBLASTS

(71) Applicant: KING'S COLLEGE LONDON, London (GB)

(72) Inventors: Fiona WATT, London (GB); Christina PHILIPPEOS, London (GB); Magnus LYNCH, London (GB)

(21) Appl. No.: 18/490,714

(22) Filed: Oct. 19, 2023

Related U.S. Application Data
(63) Continuation of application No. 16/956,500, filed on Jun. 19, 2020, now abandoned.

(30) Foreign Application Priority Data
Dec. 22, 2017 (GB) 1721738.1

(51) Int. Cl. C12N 5/077; A61K 35/36

(52) U.S. Cl. CPC C12N 5/0656; A61K 35/36; C12N 2501/115; C12N 2501/15; C12N 2501/415

(57) ABSTRACT

Provided herein is a method of sorting human dermal fibroblasts, comprising (a) providing a cell population comprising human dermal fibroblasts; and (b) separating the human dermal fibroblasts into subpopulations based on expression of one or more cell-surface markers selected from CD39, CD36 and CD26. Also provided are isolated populations of human dermal papillary fibroblasts and cosmetic and therapeutic uses thereof.

### FIELD OF THE INVENTION

The present invention relates to the field of dermatology, and in particular to the role of fibroblasts in skin conditions, ageing and wound healing. The invention relates more specifically to methods for sorting human dermal fibroblasts into subpopulations and expanding beneficial subpopulations in culture, and the uses of the sorted populations in medical and cosmetic methods and for in vitro toxicology and cosmetic screens.

### BACKGROUND OF THE INVENTION

A paradigm of the metazoan body plan is the combination of epithelial and mesenchymal elements into structured three-dimensional organs. Mammalian skin represents an archetype of this pattern: the epidermis, a stratified squamous epithelium, overlies the dermis, a mesenchymal tissue. Whilst cell lineage relationships within the epidermis and other epithelia have been studied in detail¹, the functional identity of cells comprising the dermis, in common with other mesenchymal tissues, is less well characterized².

Fibroblasts are cells that synthesize and integrate structural proteins such as collagen and elastin into the extracellular matrix of most mesenchymal tissues³⁻⁶. The dermis has distinct layers that are readily identified histologically: the papillary dermis lies closest to the epidermis, while the underlying reticular dermis is thicker and contains the bulk of the fibrillar extracellular matrix⁷. Beneath the reticular dermis lies the hypodermis, or dermal white adipose tissue^{8,9}. Other fibroblast subpopulations that have been identified in mouse and human dermis include the dermal papilla cells at the base of the hair follicle^{10,11}, the cells of the arrector pili muscle and pericytes that are in close association with blood vessels¹².

It has long been suspected that papillary and reticular fibroblasts have distinct identities⁷. In the case of mouse dermis, it has been demonstrated via lineage tracing under homeostatic conditions, during wound healing and in skin reconstitution assays that the papillary and reticular fibroblasts represent functionally distinct lineages that arise from a multipotent progenitor population during embryonic development^{13,14}. Papillary fibroblasts are required for new hair follicle formation, whereas reticular fibroblasts mediate the early events in wound repair and express so-called fibroblast activation markers such as alpha-smooth muscle actin.

Previous studies of the human dermis have mechanically separated papillary from reticular dermis with a dermatome. Analysis of explant cultures derived from these separated regions of the dermis has revealed differences in fibroblast behaviour in culture and in gene expression¹⁵⁻¹⁷. However previous attempts to define markers that distinguish fibroblasts cultured separately from the papillary and reticular dermis¹⁶ have not yielded cell surface markers that permit the prospective isolation of primary fibroblast subpopulations directly from the skin.

Accordingly there is a need for new methods for separating human dermal fibroblasts into subpopulations that are useful in the treatment of skin conditions.

### SUMMARY OF THE INVENTION

Accordingly, in one aspect the present invention provides a method of sorting human dermal fibroblasts, comprising:
(a) providing a cell population comprising human dermal fibroblasts; and
(b) separating the human dermal fibroblasts into subpopulations based on expression of one or more cell-surface markers such as CD39, CD36 and/or CD26.

In some embodiments the method may optionally further comprise a step of expanding one or more subpopulations separated in step (b), e.g. in an in vitro cell culture step. Preferably the expanded subpopulations retain their functional properties following expansion.

Preferably the human dermal fibroblasts are separated by flow cytometry. In alternative embodiments, the human dermal fibroblasts are separated using magnetic beads, columns and/or microfluidics.

In one embodiment the human dermal fibroblasts are separated from the cell population by selecting cells expressing CD90. For instance, the human dermal fibroblasts may be separated from other human dermal cells by selecting cells having a cell surface phenotype CD45− CD31− CD324− CD90+.

In one embodiment, a first subpopulation of human dermal fibroblasts is separated by selecting cells having a cell surface phenotype CD39+CD26−. The first subpopulation may comprise papillary fibroblasts.

In another embodiment, a second subpopulation of human dermal fibroblasts is separated by selecting cells having a cell surface phenotype CD39+CD26+. The second subpopulation may comprise reticular fibroblasts. Another subpopulation of human dermal fibroblasts may be separated by selecting cells having a cell surface phenotype CD39−. This subpopulation may also comprise reticular fibroblasts.

In a further embodiment, a third subpopulation of human dermal fibroblasts is separated by selecting cells expressing CD36. Preferably the third subpopulation comprises lower reticular and pre-adipocyte fibroblasts.

In another aspect, the invention provides an isolated subpopulation of human dermal fibroblasts obtained or obtainable by a method as defined above.

In another aspect, the invention provides an isolated population of human papillary fibroblasts, characterized by expression of a cell surface phenotype CD45−CD31− CD324− CD90+CD39+CD26−.

In another aspect, the invention provides an isolated population of human dermal reticular fibroblasts, characterized by expression of a cell surface phenotype CD45− CD31− CD324− CD90+CD39−.

In another aspect, the invention provides an isolated population of human dermal reticular fibroblasts, characterized by expression of a cell surface phenotype CD45− CD31−CD324−CD90+CD39+CD26+.

In another aspect, the invention provides an isolated population of human dermal pre-adipocyte fibroblasts, characterized by expression of a cell surface phenotype CD45− CD31− CD324− CD90+CD36+.

In another aspect, dermal cells that express vimentin and are CD45− CD31−CD324− and CD90− represent another potentially useful subset of cells.

In another aspect, the invention provides for in vitro expansion of fibroblast subpopulations, for example by pharmacological modulation of the Wnt or Hedgehog signalling pathways. The subpopulations may be characterised by differential expression of genes associated with Wnt signalling, extracellular matrix production/remodelling and inflammation.

In another aspect, the invention provides a pharmaceutical composition comprising an isolated population of human dermal fibroblasts as defined above, and optionally one or more pharmaceutically acceptable excipients, diluents or carriers.

In another aspect, the invention provides an isolated population of human dermal fibroblasts as defined above, for use in medicine.

In another aspect, the invention provides an isolated population of human dermal fibroblasts as defined above, for use in treating a skin disorder.

In another aspect, the invention provides an isolated population of human dermal fibroblasts as defined above, for use in promoting wound healing.

In another aspect, the invention provides an isolated population of human dermal fibroblasts as defined above, for use in treating keloidal or non-keloidal scarring, scleroderma, graft versus host disease, skin ulcers or genetic skin disorders such as epidermolysis bullosa.

In another aspect, the invention provides use of an isolated population of human dermal fibroblasts as defined above, in the prevention or treatment of a skin disorder, e.g. for promoting wound healing, treating keloidal or non-keloidal scarring, scleroderma, graft versus host disease, skin ulcers or genetic skin disorders such as epidermolysis bullosa.

In another aspect, the invention provides use of an isolated population of human dermal fibroblasts as defined above, for the preparation of a medicament for the prevention or treatment of a skin disorder, e.g. for promoting wound healing, treating keloidal or non-keloidal scarring, scleroderma, graft versus host disease, skin ulcers or genetic skin disorders such as epidermolysis bullosa.

In another aspect, the invention provides a method of preventing or treating a skin disorder (e.g. for promoting wound healing, treating keloidal or non-keloidal scarring, scleroderma, graft versus host disease, skin ulcers or genetic skin disorders such as epidermolysis bullosa) in a subject in need thereof, comprising administering a pharmaceutically acceptable amount of an isolated population of human dermal fibroblasts as defined above to the subject.

In another aspect, the invention provides a cosmetic method for preventing or treating skin ageing or scarring in a human subject, comprising administering an isolated population of human dermal fibroblasts as defined above to skin of the subject.

In one embodiment, the human dermal fibroblasts are autologous to the subject. In an alternative embodiment, the human dermal fibroblasts are allogeneic to the subject.

In a further embodiment, the fibroblast subpopulations are used for in vitro toxicology and/or cosmetics screens.

In another aspect, the invention provides a method of identifying one or more subpopulations of human dermal fibroblasts in a sample, comprising providing a sample comprising human dermal fibroblasts and determining the expression of one or more (cell-surface or intracellular) markers described herein, e.g. one or more cell-surface markers selected from CD39, CD36 and/or CD26 on said fibroblasts.

In another aspect, the invention provides use of one or more one or more (cell-surface or intracellular) markers described herein, e.g. one or more cell-surface markers selected from CD39, CD36 and/or CD26, or a ligand (e.g. antibody) thereto, for the identification of a subpopulation of human dermal fibroblasts.

In another aspect, the invention provides a kit for identifying and/or separating one or more subpopulations of human dermal fibroblasts in a sample, comprising one or more reagents specific for one or more (cell-surface or intracellular) markers defined herein, e.g. one or more cell-surface markers selected from CD39, CD36 and/or CD26. In one embodiment, the kit comprises one or more ligands (e.g. antibodies) that bind specifically to the marker(s). In another embodiment, the kit comprises one or more reagents suitable for specific amplification of a nucleotide sequence encoding the markers, e.g. nucleotide primers suitable for polymerase chain reaction (PCR) amplification of mRNA or cDNA encoding the markers.

In another aspect, the present invention provides an isolated population of human dermal papillary or reticular fibroblasts, for use in preventing or treating a skin disorder, e.g. treating a wound, promoting wound healing, promoting wound healing with reduced risk of scarring, and/or preventing or treating scarring. In one embodiment, the human dermal fibroblasts are human papillary fibroblasts, characterized by expression of a cell surface phenotype CD45− CD31− CD324− CD90+CD39+CD26−. In another embodiment, the human dermal fibroblasts are human dermal reticular fibroblasts, characterized by expression of a cell surface phenotype CD45− CD31− CD324− CD90+CD39−. In another embodiment, the human dermal fibroblasts are human dermal reticular fibroblasts, characterized by expression of a cell surface phenotype CD45− CD31− CD324− CD90+CD39+CD26+.

### BRIEF DESCRIPTION OF THE DRAWINGS

FIG. 1: Transcriptomic analysis of mouse fibroblast subpopulations. A) Isolation of mouse postnatal day 2 (P2) fibroblast subpopulations by flow cytometry. PdgfraHBeGFP+ cells were isolated and separated according to expression of Dlk1, Sca1 and CD26. B) Principal component analysis of global gene expression patterns determined by microarray hybridization (Affymetrix). Note outlier pre-adipocyte (Dlk1−Sca1+) population that clusters with the alternate pre-adipocyte (Dlk1+Sca1+) population (asterisk; also marked with asterisks in panels D and E). C) qPCR validation of marker expression on flow sorted populations. Gene expression is normalized to GAPDH and is expressed as mean±S.D. for 3 biological replicates. D) Levels of marker expression in the microarray samples; levels for each of the 3 biological replicates is shown separately. The outlier pre-adipocyte (Dlk1−Sca1+) population is indicated (asterisk). E) Heirarchical clustering of global gene expression patterns for prospectively isolated mouse P2 fibroblast subpopulations. The outlier pre-adipocyte (Dlk1−Sca1+) population is indicated (asterisk).

FIG. 2: Differential expression of genes associated with Wnt, ECM and immune signalling in mouse fibroblast subpopulations. A) Gene Ontology (GO) term analysis of differentially expressed pathways in mouse fibroblast subpopulations. B-D) Heatmaps illustrating differential expression (Affymetrix microarray) of genes implicated in (B) Wnt signalling, (C) inflammation, and (D) ECM regulation. E) Q-PCR validation of selected differentially expressed genes. F) Heatmap comparing expression (Affymetrix microarray) of genes implicated in adipogenesis. G, H) qPCR analysis demonstrating upregulation of CD36 expression in pre-adipocyte populations (G) and CD39 in papillary fibroblasts (H). Gene expression (E, G, H) is normalized to GAPDH and expressed as mean #S.D. for 3 biological replicates (*p<0.05, p<0.005, ***p<0.0005).

FIG. 3: Spatial profiling of gene expression in human dermis. A) Hierarchical clustering of gene expression patterns from papillary and reticular dermis in 3 separate individuals. Expression was quantified by RNA sequencing (blue similar, red dissimilar). B) Genes upregulated in papillary dermis (>3 fold). C) Genes upregulated in reticular dermis (>10 fold). D) Cell surface markers for which expression was at least 2-fold upregulated in papillary dermis. Expression level is indicated separately for each of the 3 biological replicates (green, high; red, low) and compared to expression in 6 different cultured fibroblast lines and 7 other cultured cell lines. E) Cell surface markers at least 2-fold upregulated in reticular dermis. F) Cell surface markers upregulated in papillary dermis that are conserved between human adult dermis and mouse P2 dermis.

FIG. 4: Immunofluorescence labelling of human dermis with antibodies to candidate fibroblast subpopulation markers identified by spatial transcriptomics. A, B) Expression of COL6A5 is restricted to the papillary dermis. The basal layer of the epidermis is labelled with anti-keratin 14. (COL6A5, green; Keratin 14, red). C,D) Expression of APCDD1 is enriched in the papillary dermis (APCDD1, green; Keratin 14, red). E, F) Expression of HSPB3 is enriched in the papillary dermis (HSPB3, green; Keratin 14, red). G,H) Expression of WIF1 is enriched in vascular structures that are more prominent in the upper dermis (WIF1, green; Keratin 14, red). I, J) Expression of CD36 is highly enriched in the hypodermis (subcutaneous fat). K,L) CD39 is enriched in the papillary dermis (CD39, green; Podoplanin, red). Scale bars: 200 μm

FIG. 5: Human dermal fibroblast subpopulations maintain functional differences in vitro. A, B) Expression of LUM and COL6A5 is enriched in CD90+ population compared to an unfractionated dermal cell suspension. Gene expression normalized to GAPDH and expressed as mean±S.D. for 3 replicates. C) lin-CD90+CD39+ and lin-CD90+CD36+ cells exhibit morphological differences in vitro but these are not consistent between different body sites and age of donor (Scale bars: 50 μm). D, E) Lin−CD90+CD39+ cells lose CD39 expression following a single passage in culture (D). However, expression of CD90 and CD36 is retained (E). F) Q-PCR showing retention of LUM and loss of COL6A5 in culture. Gene expression normalized to GAPDH and expressed as mean±S.D. for 3 biological replicates. G-L) Expression of genes implicated in Wnt signalling (G), inflammation and immunity (H) and ECM remodelling (I). Gene expression is normalized to GAPDH and is expressed as mean±S.D. for 3 biological replicates. J-N) Modulation of expression of cell surface markers in response to IFNγ stimulation in culture (blue, CD39+IFNγ; yellow, CD36+IFNγ; red, unstained control). Top row: representative flow plots. Bottom row: quantitation of data from 3 independent experiments (*p<0.05, **p<0.005, p<0.0005, ****p<0.0001).

FIG. 6: Comparison of the ability of different fibroblast subpopulations to support epidermal growth on DED. A-J) H&E staining (A-E) and immunofluorescence staining (F-J) of de-epithelialized dermal (DED) organotypic cultures without fibroblasts (A, F) or seeded with unfractionated (lin-CD90+) fibroblasts (B, G), CD90+CD39+(enriched in papillary) fibroblasts (C, H) CD90+CD39− (depleted of papillary) fibroblasts (D, I) and CD90+CD36+(preadipocyte) fibroblasts (E, J). (F-J) Keratin 14 (green) marks keratinocytes, vimentin (white) marks mesenchymal cells (fibroblasts). Experiments were repeated for a minimum of 3 biological replicates and representative images are displayed. In the case of the CD90+CD39+ cells, two of three experiments involved additional selection for CD26− cells to further enrich for the papillary cell population (CD90+CD39+CD26−). Scale bars: 200 μm. K-M) Quantification of epidermal thickness (K); density of fibroblasts within 300 μm of the epidermis (L); and relative abundance of fibroblasts at different depths from the epidermis (M).

FIG. 7: Single cell RNA sequencing of human adult dermal fibroblasts. A) Isolation of lineage (lin) negative cells and lin-CD90+ cells from human dermis. Single live cells were isolated by gating for forward scatter, side scatter and DAPI staining. Lineage (lin) negative cells were isolated by gating for CD31−CD45−ECad−. B) PCA analysis of gene expression patterns C,D) tSNE analysis of gene expression patterns (red, lin−; blue, lin−CD90+) D) Automated clustering of tSNE analysis identifies 5 dermal fibroblast subpopulations. E) Expression patterns of markers differentially expressed in each of 5 clusters (red, high expression; yellow, low expression). F) Violin plots illustrating differential expression of marker genes in each of 5 dermal fibroblast subpopulations. G-J) Immunostaining for candidate fibroblast markers in adult human skin. Scale bars: 200 μm. K-O) Expression of CD39 (K) COL6A5 (L) WNT5A (M) RSP01 (N) and LEF1 (O) in lin− CD39+CD26− and CD39− dermal fibroblasts. Gene expression is normalized to GAPDH and TBP and is expressed as mean±S.D. for 3 biological replicates (*p<0.05, **p<0.005, *** p<0.0005, ****p<0.0001).

FIG. 8: Cell surface marker screen of adult human dermal fibroblasts Isolated lineage negative cells from human dermis were analyzed using BD Lyoplate Human Cell Surface Marker Screening Panel (BD Biosciences, cat. 560747) containing 242 purified monoclonal antibodies and corresponding isotype controls. Hits identified by immunofluorescent staining; brightly stained cells indicate high expression of surface marker (**). Cell surface marker transcripts were independently observed by gene expression overlay on tSNE analysis of single cell RNA sequencing data (red, high expression; yellow, low expression).

FIG. 9 shows collagen density in wounded skin seeded with different fibroblast subpopulations, cultured ex vivo for 14 days.

FIG. 10 shows images of wounded skin seeded with different fibroblast subpopulations, cultured ex vivo for 14 days.

FIG. 11 shows images of wounded skin seeded with different fibroblast subpopulations, cultured ex vivo for 14 days.

FIG. 12 shows images of wounded skin seeded with different fibroblast subpopulations, cultured ex vivo for 14 days.

FIG. 13 shows images of collagen deposition in decellularized dermis reconstituted with different fibroblast subpopulations.

FIG. 14 shows collagen density in decellularized dermis reconstituted with different fibroblast subpopulations.

FIG. 15 shows images of collagen fibre structure in decellularized dermis reconstituted with different fibroblast subpopulations, detected using collagen hybridizing peptide (CHP).

FIG. 16 shows images of R-spondin 1 expression in the epidermis of decellularized dermis reconstituted with different fibroblast subpopulations.

FIG. 17 shows images showing keloid scar remodelling by different fibroblast subpopulations. Scale bar=2.5 mm.

FIG. 18 shows the thickness of keloid scar tissue that was decellularized, injected with different fibroblast subpopulations, and cultured in vitro for 3 weeks.

### DETAILED DESCRIPTION OF THE INVENTION

In one aspect, the present invention relates to a method of sorting human dermal fibroblasts. Thus the method may involve separating fibroblasts from human skin into one or more subpopulations, typically based on the expression of specific cell surface markers.

### Cell Population Comprising Human Dermal Fibroblasts

In one embodiment, a first step of the method comprises providing a cell population comprising human dermal fibroblasts. Typically the cell population is derived from a human skin sample, i.e. the cell population comprises human skin cells. In one embodiment the cell population is obtained by enzymatic digestion of human dermis. Thus in preferred embodiments, the cell population comprises dissociated cells, i.e. cells that have been separated from their original tissue environment and dispersed in a liquid medium.

In one embodiment, the cell population comprises a cell suspension. For instance the method may comprise digesting a human skin sample to obtain a suspension of cells dispersed in a suitable aqueous buffer. The suspension may optionally be filtered and/or centrifuged to obtain a cell pellet that is then resuspended in a desired aqueous buffer, e.g. phosphate-buffered saline (PBS).

Typically the cell population (e.g. cell suspension) comprises human dermal fibroblasts as well as other (i.e. non-fibroblast) cell types derived from human skin. In some embodiments, human dermal fibroblasts may first be separated from other human skin types, before the separation into human dermal fibroblast subpopulations takes place. In alternative embodiments, the separation into human dermal fibroblast subpopulations takes place at the same time as the separation of human dermal fibroblasts from other skin cell types, i.e. there is a single separation step in which human dermal fibroblast subpopulations are fractionated from a cell suspension comprising human dermal cells. In alternative embodiments, the fibroblasts of interest are collected by migration out of the dermis in explant culture.

### Separating Human Dermal Fibroblasts

In the method of present invention, human dermal fibroblasts are separated into subpopulations based on expression of specific cell surface markers. By this it is meant that human dermal fibroblasts are sorted or fractionated into subpopulations that express a particular cell surface phenotype, e.g. expression of particular markers and/or absence of expression of other markers. For instance, human dermal fibroblast subpopulations may be separated by selecting cells that express CD90, CD39, CD36 or CD26 individually, or by selecting human dermal fibroblasts that lack expression of CD90, CD39, CD36 or CD26 individually. In some embodiments human fibroblast subpopulations may be separated based on a combination of expression and absence of expression of two or more of the above markers.

In one embodiment, the human dermal fibroblasts are separated by flow cytometry. For instance flow cytometry may be used to separate the human dermal fibroblasts from other skin cells and/or to separate the human dermal fibroblasts into subpopulations. Fluorescence cytometry or fluorescence-activated cell sorting (FACS) is a specialized type of flow cytometry which is particularly useful for identifying and isolating cells according to surface markers, and may be used in a preferred embodiment of the invention. It provides a method for sorting a heterogeneous mixture of biological cells into two or more containers, one cell at a time, based upon the specific light scattering and fluorescent characteristics of each cell. Thus FACS provides a fast, objective and quantitative recording of fluorescent signals from individual cells as well as physical separation of cells of particular interest.

In fluorescence cytometry, a cell suspension is entrained in the centre of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell per droplet. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement, and the opposite charge is trapped on the droplet as it breaks from the stream. The charged droplets then fall through an electrostatic deflection system that diverts droplets into containers based upon their charge. In some systems, the charge is applied directly to the stream, and the droplet breaking off retains charge of the same sign as the stream. The stream is then returned to neutral after the droplet breaks off. One common way to use flow cytometry is with a fluorescently labeled antibody that binds to a target on or in a cell, thereby identifying cells with a given target. This technique can be used quantitatively where the amount of fluorescence correlates to the amount of target, thereby permitting one to sort based on relative amounts of fluorescence, and hence relative amounts of the target.

Thus in a preferred embodiment of the presence invention, the cell population comprising human dermal fibroblasts may be contacted with one or more fluorescently-labelled antibodies (e.g. suitable for use in FACS). The fluorescent-labelled antibody may bind directly to a human dermal fibroblast cell surface marker (e.g. CD90, CD39, CD36 or CD26), or may be a secondary antibody that binds to a primary antibody specific for the cell surface marker (e.g. a primary mouse IgG anti-human CD39, CD36 or CD26 antibody may bind directly to the human dermal fibroblasts, and a secondary fluorescent rat anti-mouse IgG antibody may bind to the primary antibody). Accordingly human dermal fibroblasts may be separated into subpopulations expressing a combination of markers selected from CD39, CD36 and/or CD26 using flow cytometry (e.g. FACS). Multi-colour fluorescence methods (e.g. using antibodies to different cell surface proteins labelled with fluorescent labels of a different wavelength) may be used to select human dermal fibroblasts based on expression of a combination of markers in a single FACS step.

In alternative embodiments, the human dermal fibroblast subpopulations may be separated by any other method suitable for separating cells based on expression of cell-surface markers. For instance, suitable methods may involve the use of magnetic beads, columns and/or microfluidics. In such methods, a ligand (e.g. an antibody) specific for the cell-surface marker may be immobilised on a solid phase (e.g. magnetic bead), and the cell population comprising human dermal fibroblasts in suspension contacted thereto. Human dermal fibroblasts expressing the marker of interest then bind to and are retained on the solid phase. Other human dermal fibroblasts lacking the marker do not bind to the solid phase and may thus be separated from the desired subpopulation by washing out the supernatant. The desired human fibroblast subpopulation may then be eluted from the solid phase if required.

### Human Dermal Fibroblasts

Human dermal fibroblasts may be distinguished from other human dermal cell types based on the expression of characteristic cell surface markers. For instance, in one embodiment CD90 expression may be used as a marker of human dermal fibroblasts. In other embodiments, human dermal fibroblasts may be identified as lin− CD90+ cells or lin− CD90− cells. Lin− CD90− cells present as a population of cells with mixed characteristics of pre-adipocytes, fibroblasts and monocyte/macrophages. The lin− cell surface phenotype refers to cells lacking expression of CD45, CD31 and CD324. CD45 expression may be indicative of immune cells, CD31 is typically expressed on endothelial cells and CD324 may be expressed on keratinocytes. Therefore human dermal fibroblasts may be identified in one embodiment as e.g. CD45− CD31− CD324− CD90+ cells.

Thus in one embodiment of the invention, human dermal fibroblasts may first be separated from other human skin cell types by selecting CD90+(e.g. CD45-CD31− CD324− CD90+) cells. The total human dermal fibroblast population may then be separated into subpopulations based on e.g. CD39, CD36 and/or CD26 expression. Alternatively, human dermal fibroblast subpopulations may be separated from other human skin cell types in a single step, e.g. by selecting CD90+CD39+CD26− (CD45− CD31− CD324− CD90+CD39+CD26−) cells, CD90+CD39− (CD45− CD31− CD324− CD90+CD39−) cells, CD90+CD36+(CD45− CD31− CD324− CD90+CD36+) cells and so on. These selections may be performed, for instance, in one or more flow cytometry steps, e.g. using particular combinations of antibodies.

In a further embodiment, fibroblasts with the desired characteristics are selectively expanded in culture by pharmacological modulation of signalling pathways such as Wnt or Hedgehog. This may involve prior FACS enrichment or plating of unfractionated cells, either in the form of single cells or explants.

### Markers

In embodiments of the present invention, expression of one or more cell surface markers may be determined. In general, cells are determined as being either positive (+) or negative (−) for expression of each cell surface marker. By positive (+) it is typically meant that the cell expresses at least a minimum (e.g. detectable) level of the cell surface marker. For example, a cell may be considered to be positive (+) for the cell surface marker if a fluorescently-labelled antibody specific for the cell surface marker is bound to the cell, e.g. in sufficient amounts that are detectable by flow cytometry (FACS). Similarly, a cell may be considered to be negative (−) for a cell surface marker if it expresses the marker below a minimum (e.g. detectable) level. Accordingly a cell surface phenotype of a cell population may be designated based on expression and/or absence of expression of particular markers, e.g. CD45− CD90+CD39−.

Antibodies to the markers discussed herein suitable for use in flow cytometry are known and typically available from commercial sources, or may be generated by known techniques such as immunization of experimental animals with a suitable antigen. Suitable antibodies are described in the examples below.

### CD26

CD26 is a cell membrane glycoprotein and serine exopeptidase expressed on the surface of various cell types. CD26 is also known as dipeptidyl peptidase-4 and adenosine deaminase complexing protein 2. The amino acid sequence of human CD26 is disclosed in, for example, database accession nos. P27487 (UniProt) and NP_001926 (NCBI RefSeq). CD26 may be detected by flow cytometry and CD26+ and CD26− cells sorted e.g. as disclosed in Kelemen et al., Am J Clin Pathol. 2008 January; 129(1):146-56.

### CD36

CD36 is a membrane protein that is a member of the class B scavenger receptor family. CD36 is also known as platelet glycoprotein 4, fatty acid translocase, scavenger receptor class B member 3 (SCARB3), glycoprotein 88 (GP88), glycoprotein IIIb (GPIIIB), or glycoprotein IV (GPIV). The amino acid sequence of human CD36 is disclosed in, for example, database accession nos. P16671 (UniProt) and NP_000063, NP_001001547, NP_001001548, NP_001120915 and NP_001120916 (NCBI RefSeq). CD36 may be detected by flow cytometry and CD36+ and CD36− cells sorted e.g. as disclosed in Cserti-Gazdewich et al., Cytometry B Clin Cytom. 2009 March;76(2):127-34.

### CD39

CD39 is a cell surface-located ectonucleotidase also known as ectonucleoside triphosphate diphosphohydrolase-1 (ENTPD1). The amino acid sequence of human CD39 is disclosed in, for example, database accession nos. P49961 (UniProt) and NP_001091645, NP_001157650, NP_001157651, NP_001157653 and NP_001157654 (NCBI RefSeq). CD39 may be detected by flow cytometry and CD39+ and CD39− cells sorted e.g. as disclosed in Mandapathil et al., Journal of Immunological Methods, Volume 346, Issues 1-2, 31 Jul. 2009, Pages 55-63.

### CD90

CD90 is an N-glycosylated, glycophosphatidylinositol (GPI) anchored conserved cell surface protein also known as Thy-1 that is a member of the immunoglobulin superfamily. The amino acid sequence of human CD90 is disclosed in, for example, database accession nos. P04216 (UniProt) and NP_001298089, NP_001298091 and NP_006279 (NCBI RefSeq). CD90 may be detected by flow cytometry and CD90+ and CD90− cells sorted e.g. as disclosed in Nakamura et al., British Journal of Dermatology 154(6):1062-1070 (2006).

### CD45

CD45 is also known as leukocyte common antigen (LCA) and protein tyrosine phosphatase, receptor type, C (PTPRC). CD45 is a membrane glycoprotein expressed on almost all hematopoietic cells except mature erythrocytes. The amino acid sequence of human CD45 is disclosed in, for example, database accession nos. P08575 (UniProt) and NP_001254727, NP_002829 and NP_00563578 (NCBI RefSeq). CD45 may be detected by flow cytometry and CD45+ and CD45− cells sorted e.g. as disclosed in Janossy et al., Clinical and Vaccine Immunology 9(5):1085-1094 (2002).

### CD31

CD31 is a cell surface protein also known as platelet endothelial cell adhesion molecule 1 (PECAM-1). The amino acid sequence of human CD31 is disclosed in, for example, database accession nos. P16284 (UniProt) and NP_000433 (NCBI RefSeq). CD31 may be detected by flow cytometry and CD31+ and CD31-cells sorted e.g. as disclosed in Khan et al., Cytometry 64B(1):1-8 (2005) and Mock et al., Mucosal Immunology (2014) 7, 1440-1451.

### CD324

CD324 a cell-cell adhesion glycoprotein also known as cadherin-1, E-cadherin, CDH1 or uvomorulin. The amino acid sequence of human CD324 is disclosed in, for example, database accession nos. P112830 (UniProt) and NP_001304113, NP_001304114 and NP_001304115 (NCBI RefSeq). CD324 may be detected by flow cytometry and CD324+ and CD324− cells sorted e.g. as disclosed in U.S. Pat. No. 9,534,058.

### Additional Markers Identified by Transcriptional Profiling of Human Dermis

In the examples described herein, single cell RNA sequencing of fibroblasts from human dermis was used to identify additional markers of fibroblast subpopulations. These additional markers may comprise intracellular as well as cell surface markers, and may be used to further characterise the fibroblast subpopulations. For instance, in some embodiments flow-sorted fibroblast subpopulations (before or after an in vitro expansion step in culture) may be analysed for expression of one or more of the additional markers, e.g. as a validation step to confirm the homogeneity of the subpopulation and/or retention of characteristic properties of the subpopulation. Analysis of the additional markers may be performed by any suitable method for detection of the RNA and/or corresponding polypeptide sequences in the subpopulation of cells, e.g. RT-PCR, RNA-Seq, gene expression arrays, Northern/Western blots, ELISA and immunocytochemistry. Expression of these additional markers may identify further subpopulations of fibroblasts that are not necessarily restricted by spatial compartmentalization within the dermis, e.g. fibroblasts expressing these additional markers may be found within more than one region of the skin.

In one embodiment, the fibroblasts or subpopulation(s) thereof may be analysed for expression of COL6A5, COL23A1 and/or HSPB3. Typically expression of these markers is associated with papillary fibroblasts. COL6A5 is the α5 chain of collagen VI, and in particular represents a robust marker for dermal fibroblasts. Additional markers of papillary fibroblasts may include WNT5a, RSPO1 and LEF1. The database accession numbers for sequences encoding these markers are shown in Table 2 below.

In another embodiment, the fibroblasts or subpopulation(s) thereof may be analysed for expression of CD70 (see e.g. database accession numbers NM_001252.4 and NP_001243.1) and/or CD34 (see database accession numbers NM_001025109.1 and NP_001020280.1). In some cases expression of these markers is associated with reticular fibroblasts, e.g. cells having a cell surface phenotype CD39+CD26+.

In another embodiment, the fibroblasts or subpopulation(s) thereof may be analysed for expression of RGS5 (regulator of G protein signaling 5). See database accession numbers NM_001195303.2, NP_001182232.1, NM_001254748.1 NP_001241677.1, NM_001254749.1, NP_001241678.1, NM_003617.3 and NP_003608.1 for examples of mRNA and polypeptide sequences encoding RGS5. Expression of this marker may be associated with pericytes.

In another embodiment, the fibroblasts or subpopulation(s) thereof may be analysed for expression of MFAP5 (microfibril associated protein 5) and/or PRG4 (proteoglycan 4). See database accession numbers NM_001297709.1 and NP_001284638.1 for examples of mRNA and polypeptide sequences encoding MFAP5. See database accession numbers NM_001127708.2 and NP_001121180.2 for examples of mRNA and polypeptide sequences encoding PRG4.

In further embodiments, the fibroblasts may be analysed for expression of one or more pan-fibroblast markers, e.g. PDGFRα (platelet-derived growth factor receptor-alpha), PDGFRβ (platelet-derived growth factor receptor-beta), decorin or lumican. Additional markers found in fibroblast populations that may be analysed include CD9, CD11a, CD29, CD44, CD47, CD59, CD73, CD81, CD87, CD105, CD141, CD142, CD147, HLA-A, HLA-B, HLA-C, HLA-DR, HLA-DP, HLA-DQ and disialoganglioside GD2. Such cell-surface markers may also be used in sorting methods (e.g. flow cytometry) as described above.

### Human Dermal Fibroblast Subpopulations

In the methods described herein, human dermal fibroblasts may be sorted into subpopulations based on the expression of any combination of the markers described herein, including any of the additional markers mentioned above. An isolated subpopulation of human dermal fibroblasts may be characterized by expression of a particular cell surface phenotype, e.g. CD45-CD90+CD39+CD26−. By “isolated” it is typically meant that the population of cells is separated from its natural environment, e.g. the subpopulation of human dermal fibroblasts is separated from the total population of human dermal fibroblasts and/or other dermal cells in the source sample (e.g. a human skin sample).

In one embodiment, the isolated cell population is characterized by expression of a cell surface phenotype CD90+CD39+CD26−, preferably CD45− CD31− CD324− CD90+CD39+CD26−. Typically this subpopulation comprises human papillary fibroblasts.

In another embodiment, the isolated cell population is characterized by expression of a cell surface phenotype CD90+CD39−, preferably CD45− CD31− CD324− CD90+CD39−. Typically this subpopulation comprises human dermal reticular fibroblasts.

In another embodiment, the isolated cell population is characterized by expression of a cell surface phenotype CD90+CD39+CD26+, preferably CD45− CD31− CD324− CD90+CD39+CD26+. Typically this subpopulation comprises human dermal reticular fibroblasts.

In another embodiment, the isolated cell population is characterized by expression of a cell surface phenotype CD90+CD36+, preferably CD45− CD31− CD324− CD90+CD36+. Typically this subpopulation comprises human dermal pre-adipocyte fibroblasts.

In another embodiment, the isolated cell population is characterized by expression of a cell surface phenotype CD90+CD39+CD26−, preferably CD45-CD31− CD324− CD90+CD39+CD26−. Typically this subpopulation comprises human papillary fibroblasts.

In another embodiment, the isolated cell population is characterized by being vimentin+ and lin− CD90− but expressing CD74 (macrophage inhibitory factor receptor), HLA-DR and/or CLDN5 and TEK (TIE2). Typically, this subpopulation comprises cells with the ability to contribute to pre-adipocyte fibroblasts and pericytes.

### Expansion of Isolated Fibroblast Subpopulations

In some embodiments the method may optionally further comprise a step of expanding one or more subpopulations separated in step (b), e.g. in an in vitro cell culture step. The isolated cell populations may be expanded using known cell culture methods, e.g. as described in Driskell et al. J Invest Dermatol. 2012 April; 132(4): 1084-1093. Preferably the expanded subpopulations retain their functional properties following expansion.

In some embodiments, the properties of the fibroblast subpopulations may be maintained or modified in culture by, for example, pharmacological modulation of the Wnt, TGFbeta, FGF or Hedgehog signalling pathways. Examples of suitable compounds for inhibiting such pathways are described in, for example, Lichtenberger et al., Epidermal b-catenin activation remodels the dermis via paracrine signalling to distinct fibroblast lineages, Nature Communications (2016) 7:10537 |DOI: 10.1038/ncomms10537. For instance, PD173074 (Tocris) is a suitable FGFR inhibitor, IPI4182 (Infinity Pharmaceuticals) inhibits Hedgehog signalling and RepSox (Sigma Aldrich) and SB431542 (Tocris) may be used as TGF-beta inhibitors. Agonists of these pathways that may be used include SUN 11602 (a basic fibroblast growth factor mimetic); TGFbeta; 20(S)-Hydroxycholesterol and SAG 21k (agonists of the Hedgehog pathway); and purified Wnt protein or inhibitors of GSK3 such as CHIR99021 and LiCl (activators of WNT signalling).

### Therapeutic and Cosmetic Applications

The isolated human dermal fibroblast populations described herein may be used in various cosmetic and therapeutic methods. In some embodiments, the isolated cell populations may be expanded ex vivo before administration to a subject. In other embodiments, the isolated cell populations may be administered directly to a subject, i.e. without ex vivo expansion. In further embodiments, the cells may be combined with an acellular scaffold, whether biological (e.g. decellularised human dermis) or inert (e.g. hydrogel). In each case, the isolated cell population may be administered to a subject from which the original cell population was obtained (i.e. in an autologous cell therapy), or the isolated cell population may be administered to a different subject (i.e. in an allogeneic cell therapy).

The isolated human dermal fibroblast populations may be used to treat various skin disorders. For example, the isolated human dermal fibroblast populations may be used to promote wound healing, or to treat keloidal or non-keloidal scarring, scleroderma, graft versus host disease, skin ulcers or genetic disorders such as epidermolysis bullosa. The isolated human dermal fibroblasts subpopulations may be used alone or in combination. For instance, papillary fibroblasts (e.g. CD90+CD39+CD26− cells) may be used alone or in combination with another subpopulation of fibroblasts (e.g. any one of the isolated fibroblast subpopulations defined herein by a combination of cell surface markers, reticular fibroblasts, pre-adipocyte fibroblasts or vimentin+ lin− CD90− cells. In one embodiment, a human dermal papillary fibroblast subpopulation is used in such methods, e.g. an isolated population characterized by a cell surface phenotype CD45− CD31− CD324− CD90+CD39+CD26−.

The isolated human dermal fibroblast populations may also be used in various cosmetic methods, e.g. for preventing or treating skin ageing, wrinkles or scarring in a human subject. In one embodiment, the isolated human dermal fibroblast populations may be used to reduce fibrosis in the skin of a subject. For instance, the cell populations may be used to modify facial contour deformities such as nasolabial folds, glabellar crease, deep wrinkles of the forehead, and acne scars. In one embodiment, a human dermal papillary fibroblast subpopulation is used in such methods, e.g. an isolated population characterized by a cell surface phenotype CD45− CD31− CD324− CD90+CD39+CD26−.

The isolated human dermal fibroblast subpopulations described herein may be administered to a subject using known methods, e.g. as performed in existing clinical trials for total human dermal fibroblast therapies. For instance, the cell populations described herein may be administered to a subject using methods described in clinical trials NCT01743053, NCT01115634, NCT02493816 and NCT00642642 (available from clinicaltrials.gov). Methods for performing fibroblast-based cell therapies are also reviewed in, for example, Leavitt T, et al., Scarless wound healing: finding the right cells and signals, Cell Tissue Res. 2016 September; 365(3):483-93 and Weiss R A, Autologous cell therapy: will it replace dermal fillers? Facial Plast Surg Clin North Am. 2013 May; 21(2):299-304. An example of an FDA-approved fibroblast therapy is LAVIV® (azficel-T) from Fibrocell Technologies, Inc., which comprises autologous fibroblasts suspended in DMEM. Fibroblast subpopulations according to the present invention may be administered in an analogous manner.

The isolated human dermal fibroblast subpopulations may be combined with any suitable pharmacologically acceptable excipients, diluents or carriers for administration to a subject. Typically the cell populations are formulated in a liquid medium, e.g. a suitable sterile buffer solution such as buffered Dulbecco's Modified Eagles Medium (DMEM). Pharmaceutical formulations comprising the isolated human dermal fibroblast subpopulations may be administered by any suitable route, preferably by injection, e.g. by intradermal injection into the skin of a subject.

A skilled person may determine a suitable dosage of the cell populations depending on the nature of the condition to be treated, the subject and so on. For instance, a single dose may comprise 10,000 to 100 million cells, preferably about 20 million cells. Typically such a dose is administered (e.g. by injection) in 0.1 to 10 ml of solution, e.g. about 1 ml of a suitable sterile buffer solution.

The efficacy of fibroblast subpopulations in the above therapeutic and cosmetic applications may be confirmed in preclinical and clinical studies, including in in vitro and in vivo animal models. For instance, suitable models of keloid scarring based on analysing the activity of keloid-derived fibroblasts, including a keloid implantation animal model, are described in J. Liu, et al., Human adipose tissue-derived stem cells inhibit the activity of keloid fibroblasts and fibrosis in a keloid model by paracrine signaling, Burns (2017) [Epublication, http:/dx.doi.org/10.1016/j_burns.2017.08.017]. The effect of fibroblast subpopulations according to the present invention in such models may also be determined. The treatment of keloid scarring in general is described in Ogawa R. Keloid and Hypertrophic Scars Are the Result of Chronic Inflammation in the Reticular Dermis. Int J Mol Sci. 2017 Mar. 10; 18(3). Thus suitable studies to determine the effects of fibroblast subpopulations in the treatment of keloid scarring may include in vitro keloid explants with fibroblast subpopulation injections, collagen gel contraction assays, and proliferation assays of keloid fibroblasts treated with cultured medium from fibroblast subpopulations.

The effects of fibroblast subpopulations on wound healing may also be determined in suitable in vitro and in vivo models. For instance migration assays, in vitro explant cultures of biopsy wounds, extracellular matrix secretion assays, and in vivo excision wound healing models may be performed using fibroblast subpopulations in order to determine their effect.

### Screening Methods

In a further embodiment, the fibroblast subpopulations described herein are used for in vitro toxicology and cosmetics screens that would enable skilled persons to avoid testing compounds on living animals. For instance, active agents may be tested in vitro on the fibroblast subpopulations, and their effects determined.

Accordingly in one aspect the present invention further provides a method for screening for a cosmetic or therapeutic agent, comprising applying the agent to an isolated fibroblast subpopulation as described herein in vitro, and determining one or more effects thereof. For instance, the method may involve determining cell viability, expression of characteristic markers or functional properties (e.g. ability to repopulate decellularised human dermis) of the fibroblast subpopulation in the presence of the active agent. High throughput screening methods may be used to identify agents capable of enhancing desirable functional properties of fibroblast subpopulations without significant toxic effects.

### Identifying Human Dermal Fibroblast Subpopulations

In further embodiments, any of the cell-surface and/or intracellular markers described herein may be used for identifying subpopulations of human dermal fibroblasts in a sample. Typically such methods involve the determination of (e.g. mRNA and/or protein) expression of one or a combination of markers in a subset of human dermal fibroblasts in the sample.

For instance, the method may comprise determining expression of one or more cell-surface markers selected from CD39, CD36 and/or CD26 on said fibroblasts. In another embodiment, the method may comprise determining expression of one or more intracellular markers, e.g. one or more of the additional markers identified by transcriptional profiling of human dermis described above and in the examples.

Expression of such markers in the sample may be determined by any suitable method for detection of the mRNA and/or corresponding polypeptide sequences in the subpopulation of cells, e.g. RT-PCR, RNA-Seq, gene expression arrays, Northern/Western blots, ELISA and immunocytochemistry. Preferably the method comprises determining a plurality of such markers, e.g. by RT-PCR or ELISA, in order to characterise the human dermal fibroblast subpopulations more fully.

For instance, in one embodiment papillary fibroblasts may be identified by expression of COL6A5, COL23A1 HSPB3, WNT5a, RSPO1 and/or LEF1, e.g. by detecting mRNA encoding one or more of these markers. Reticular fibroblasts may be identified by expression of CD70 and/or CD34.

### Kits

Also provided herein are kits for identifying and/or separating one or more subpopulations of human dermal fibroblasts in a sample. Such kits may comprise reagents specific for one or more markers defined herein. Preferably the kit comprises reagents specific for a plurality of markers. Suitable reagents include ligands (e.g. antibodies) that bind specifically to the marker(s), or reagents such as oligonucleotide primers that direct specific amplification of marker sequences, e.g. mRNA or cDNA encoding the markers. The kits may comprise further reagents suitable for performing the detection method, e.g. reagents for ELISA assays such as secondary antibodies, buffer solutions or fluorescent labels or reagents for performing RT-PCR such as reverse-transcriptase, Taq polymerase, deoxyribonucleotides (dNTPs) and a suitable buffer.

In a preferred embodiment, the kit comprises a combination of reagents specific for CD39, CD36 and/or CD26. For instance, the kit may comprise antibodies or primers specific for CD39, CD36 and CD26. The kit may optionally further comprise reagents (e.g. antibodies or primers) specific for CD90, CD45, CD31 and/or CD324.

In another embodiment, the kit comprises a combination of reagents specific for markers of papillary fibroblasts. For instance, the kit may comprise one or more nucleotide primers specific for COL6A5, COL23A1 HSPB3, WNT5a, RSP01 and/or LEF1. Preferably the kit comprises at least two, three, four, five or six primer pairs, each primer pair being suitable for amplification of COL6A5, COL23A1 HSPB3, WNT5a, RSP01 and/or LEF1 mRNA or cDNA. Examples of primers pairs suitable for amplification of papillary fibroblast specific markers are shown in Table 1 below:

TABLE 1

| | Forward Primer | Reverse Primer |
|---|---|---|
| Gene | 5′-3′ | 5′-3′ |
| Lef1 | ATCACACCCGTCACACATCC | GGGGTGATCTGTCCAACACC |
| | (SEQ ID NO: 1) | (SEQ ID NO: 2) |
| RSPO1 | TCTGCTCTGAAGTCAACGGC | GCCTCACAGTGCTCGATCTT |
| | (SEQ ID NO: 3) | (SEQ ID NO: 4) |
| Wnt5a | CGCGAAGACAGGCATCAAAG | CGTATGTGAAGGCCGTCTCG |
| | (SEQ ID NO: 5) | (SEQ ID NO: 6) |
| Col6A5 | TGCGCTGAACCTTCGACTG | AGCGTGGAAATTGTCTGTTC |
| | (SEQ ID NO: 7) | TG (SEQ ID NO: 8) |
| Col23A1 | AGGACAAGATGGAGCTGCTG | ATCTTTCCCAGTGTCGCCAG |
| | (SEQ ID NO: 9) | (SEQ ID NO: 10) |
| HSPB3 | GGGCACGGCTATAAACCACT | TCCTTCTGCCAACCTTCCAC |
| | (SEQ ID NO: 11) | (SEQ ID NO: 12) |

Thus in particular embodiments, the kit may comprise one or more primers having the sequence of one or more of SEQ ID NO:s 1 to 12. Preferably the kit comprises at least two, four, six, eight, ten or twelve of SEQ ID NOs 1 to 12.

A skilled person can easily identify alternative primer sequences suitable for specific amplification of the desired markers, using known methods. The nucleotide and amino acid sequences of the markers described herein, including various polymorphic variants thereof, are available from publicly-accessible sequence databases, as shown for example in Table 2 below:

TABLE 2

| | | Database accession numbers |
|---|---|---|
| Gene | Full name | (human mRNA sequence) |
| Lef1 | Lymphoid enhancer binding factor 1 | NM_016269.4, |
| | | NM_001166119.1, |
| | | NM_001130714.2, |
| | | NM_001130713.2 |
| RSPO1 | R-spondin-1 | NM_001038633.3, |
| | | NM_001242908.1, |
| | | NM_001242909.1, |
| | | NM_001242910.1 |
| Wnt5a | wingless-type MMTV integration site family, | NM_001256105.1 |
| | member 5A | NM_003392.4 |
| Col6A5 | collagen type VI alpha 5 chain | NM_001278298.1 |
| | | NM_153264.6 |
| Col23A1 | collagen type XXIII alpha 1 chain | NM_173465.3 |
| HSPB3 | heat shock protein family B (small) member 3 | NM_006308.2 |

In further embodiments, the kit may comprise reagents (e.g. antibodies or primers) specific for any combination of the following markers, e.g. at least two, three, four, five, six, seven, eight, nine or ten of the following markers: CD90, CD70, CD34, RGS5, PRG4, MFAP5, vimentin, lumican or decorin.

The invention will now be described by way of example only, with reference to the following non-limiting embodiments.

### EXAMPLES

### Example 1

Using a combination of transcriptional profiling of flow sorted mouse fibroblast subpopulations, spatial profiling of microdissected human dermis and single cell RNA sequencing of primary human dermal fibroblasts, we define markers that permit the prospective isolation of human dermal fibroblast subpopulations. We further demonstrate that prospectively isolated fibroblasts from the upper and lower dermis exhibit distinct properties, including differences in Wnt signalling, differential responsiveness to interferon stimulation and the ability to support the development of a fully stratified human epidermis in three dimensional organotypic cell culture. Our findings are of considerable relevance to the investigation of wound healing and human disease states characterized by excessive fibrosis and offer cell surface markers that can facilitate the ex vivo expansion or in vivo ablation of specific fibroblast subpopulations for therapeutic applications.

### Materials and Methods

### Histology

Surplus surgical waste skin was obtained from consenting patients undergoing plastic surgery. This work was ethically approved by the National Research Ethics Service (UK) (HTA Licence No: 12121, REC No: 14/NS/1073). For histology, tissue samples were embedded in optimal cutting temperature compound (OCT, Life Technologies) and stored at −80° ° C. 10-16 mm sections were cut using a Thermo Cryostar Nx70 (Thermo Fisher Scientific). Sections were fixed in 4% paraformaldehyde, blocked with a solution of 10% donkey serum, 0.1% fish skin gelatin, 0.1% Triton X-100, and 0.5% Tween-20 (all from Sigma-Aldrich) in PBS and labelled with primary antibodies diluted in blocking buffer overnight at 4° C. Sections were washed with PBS, and then labelled with secondary antibodies and DAPI for 1 h at room temperature, washed with PBS and mounted with Fluorescence Mounting Medium (DAKO).

### Antibodies

The following primary antibodies were used for immunofluorescence labelling and flow cytometry: anti-mouse CD26 PerCP-Cy5.5 (eBioscience 45-0261-80), anti-human CD26 PE-Cy5 (Biolegend 302708), anti-mouse CD133 PE (eBioscience 12-1331-80), anti-mouse CD133 APC (eBioscience 17-1331-81), anti-mouse CD140a APC (eBioscience 17-1401-81), anti-mouse Ly-6A/E AF700 (eBioscience 56-5981-82), anti-mouse Dlk1 PE (MBL Int/Caltag medsystems D187-5), anti-human CD31 PE (eBioscience 12-0319-41), anti-human CD31 APC-Cy7 (Biolegend 303119), anti-human CD36 FITC (eBioscience11-0369-41), anti-human CD36 PE (Biolegend 336206), CD39 (eBioscience 14-0399-82), anti-human CD39 PE (eBioscience 1112-0399-41), anti-human CD39 APC (Biolegend 328210), anti-human CD45 AF700 (eBioscience 111256-9459-41), anti-human CD45 APC-Cy7 (Biolegend 368516), anti-human CD90 PE (eBioscience 12-0909-41), anti-human CD90 APC (Biolegend 328114), anti-human CD324 PerCP/Cy5.5 (Biolegend 324113), Anti-human CD86 PE (Biolegend 305405), Anti-human CD40 APC/Cy7 (Biolegend 334323), Anti-human HLA-DR Pacific Blue™ (Biolegend 327016), Anti-human CD80 Brilliant Violet 605™ (Biolegend 305225), Anti-Human CD274 (PD-L1, B7-H1) PE-Cyanine7 (eBioscience 25-5983-41), K14 (Cambridge Bioscience 906001), PDGFR-α (R&D Systems AF307-NA), Vimentin (Cell Signaling 5741S), Podoplanin (R&D Systems AF3244), Col6A5 (Abcam Ab122836), APCDD1 (Abcam Ab171851), HSPB3 (Abcam Ab150844), WIF-1 (R&D Systems MAB134), MFAP5 (Atlas Antibodies HPA010553), and PRG4 (Atlas Antibodies HPA028523).

The following secondary antibodies used: anti-mouse AF555 (Invitrogen A31570), anti-mouse AF647 (Invitrogen A31571), anti-goat AF488 (Invitrogen A110550), anti-goat AF555 (Invitrogen A21432), anti-rabbit AF488 (Invitrogen A21206), anti-rabbit AF555 (Invitrogen A31572), anti-rat AF488 (Invitrogen A21208), anti-rat AF555 (Invitrogen A21434) and anti-chicken AF488 (Invitrogen A11039).

### Microscopy

Photomicrographs were taken using a Leica DM IL LED Tissue culture microscope. Confocal microscopy was performed with a Nikon A1 Upright Confocal microscope using 10× or 20× objectives. Imaging of H&E stained sections was performed using a Hamamatsu NanoZoomer slide scanner (Hamamatsu). Image processing was performed with Nikon Elements, Image J (Fiji), Photoshop CS6 (Adobe) and Icy software.

### Isolation of Neonatal Mouse Fibroblasts

P2 dermis was harvested as described previously⁶². The limbs, tail and the head were removed. An anterior to posterior incision in the ventral skin was made, and the skin was separated from the carcass. Skin was incubated for 1 h at 37° C. in a solution of Trypsin/EDTA (Sigma-Aldrich) and Dispase (Sigma-Aldrich) (50:50) after which the epidermis was peeled off and discarded. The dermis was minced and incubated for 1 h at 37° C. in 0.25% collagenase in FAD basal medium (Gibco). The resulting cell suspension was filtered through a 70 μm cell strainer (SLS), and centrifuged at 1800 rpm for 4 min at 25° C. The supernatant was removed and the pellet was washed three times with PBS. Finally the pellet was resuspended in Amniomax medium (Gico) and cells were used for flow cytometry and RNA extraction.

### Isolation of Adult Human Fibroblasts

Human adult surgical waste skin was cut into 5 mm diameter pieces and incubated with dispase for 1 h at 37° C. The epidermis was peeled off and discarded and the dermis was digested overnight at 37° C. using enzymes from a whole-skin dissociation kit (Miltenyi). The resulting cell suspension was filtered through a 70 μm cell strainer, and centrifuged at 1500 rpm for 10 min at 4° C. The supernatant was removed and the pellet was washed once with PBS at 1500 rpm for 4 min at 4° C. The pellet was resuspended in PBS+1% FCS for flow cytometry or lysis buffer containing 2-mercaptoethanol (Qiagen) for RNA extraction.

### Cell Culture

Human adult dermal fibroblasts were cultured in Dulbecco's modified eagle medium (DMEM)+10% (v/v) FBS, 2 mM L-glutamine, and 100 U/ml penicillin, 100 μg/ml streptomycin (Gibco) or Amniomax C100 medium with Amniomax C100 supplement (Gibco). Culture flasks were incubated at 37° C. in a humidified atmosphere with 5% CO₂ and passaged every 3-5 days, when 80% confluent. Cells were used between passages 1-6 for all studies. Stock cultures of primary normal human keratinocytes (NHKs, strain km) were obtained from surgically discarded foreskin and grown on 3T3-J2 feeder cells. NHKs were used for DED experiments between passages 2-5. 3T3-J2 fibroblasts were originally obtained from Dr James Rheinwald (Department of Dermatology, Harvard Skin Research Centre, USA), not authenticated. All cell stocks were routinely tested for mycoplasma contamination and were negative. NHKs were cultured in complete FAD medium, containing 1 part Ham's F12, 3 parts DMEM, 10⁻⁴ M adenine, 10% (v/v) FBS, 0.5 μg ml⁻¹ hydrocortisone, 5 μg ml⁻¹ insulin, 10⁻¹⁰ M cholera toxin and 10 ng ml⁻¹ EGF, on mitotically inactivated 3T3-J2 cells as described previously^{63,64}. For INF-γ-stimulation assays, human dermal fibroblasts were stimulated with 1000 U/ml INF-γ (Sigma-Aldrich) for 72 h in growth medium, prior to harvesting for analysis by flow cytometry.

De-epidermised dermis (DED) was prepared as described previously⁶⁵. Briefly, adult human skin was divided into 1-2 cm², heated at 52° C. for 20 min and the epidermis separated from the dermis with forceps. The dermis was depleted of cells by at least ten freeze-thaw cycles, and irradiated once with 60 Gy. Before fibroblasts were seeded onto DEDs, the tissue was placed into 6-well hanging cell culture inserts (Millipore) and equilibrated with DMEM. Fibroblasts, 5×10⁵ cells/DED, were injected into the DED using U-100 insulin syringes (BD) from the epidermis surface, and then incubated for 72 h completely submerged in DMEM. Medium was changed to FAD medium with an air-liquid interface and 1×10⁶ keratinocytes were seeded on top of the DED. DEDs were maintained in culture with FAD medium and an air-liquid interface for 3 weeks with media changes every 48 h.

### Flow Cytometry

Disaggregated dermal cells were labeled with antibodies in PBS+1% FCS for 45 min at 4° C. DAPI was used to exclude dead cells. Fluorescence minus one (FMO) controls were used during the experimental set up. Following incubation, cells were centrifuged at 1500 rpm for 4 min at 4° C., and washed three times in PBS+1% FCS. Pellets were resuspended in PBS+1% FCS and filtered through a 50 μm cell strainer. Data acquisition was performed using the BD FACSCanto II fluidics and LSRFortessa cell analyser systems. Cell sorting was performed on the BD FACSAria™ II and BD FACSAria™ III Fusion cell sorters. For gate setting and compensation, unlabelled, single-labeled cells and compensation beads (BD) were used as controls. Data analysis was performed using FlowJo software version 7.6.5 (Tree Star, Ashland, OR).

### Quantitative RT-PCR

Total RNA was isolated using the Qiagen RNeasy mini kit (Qiagen) and cDNA was synthesised using the Quanti Tect Reverse Transcription kit (Qiagen) or Superscript III First-Strand Synthesis (Thermofisher). Additional RNAse H treatment was completed at the end of the reaction, by adding 1 μl of RNAse H per 30 μl reaction, for 20 min in PCR block at 37° C.

RT-qPCR reactions were performed on CFX384 Real-Time System (Bio-Rad) using the standard protocols for TaqMan Fast Universal PCR Master Mix with TaqMan probes, or using SYBR-Green Master Mix (Life Technologies) using qPCR primers (published or designed with Primer3). Values were normalised to GAPDH, 18S or TBP expression levels using the Delta CT method. Each reaction was completed with at least biological triplicates unless otherwise stated. The following TaqMan probes were used: CD36 Mm01135198_m1; AKAP12 Mm00513511_m1; CHODL Mm00507273_m1; Mouse GAPDH Endogenous Control (4352339E), CD39 Mm00515447_m1; Akr1c18 Mm00506289_m1., IL6 Mm00446190_m1, NRK Mm00479081 ml.

### Affymetrix Microarrays

cDNA was fragmented and labeled with the Affymetrix GeneChip® Labeling Kit. The labeled DNA target and fragmented cDNA were hybridized on Mouse Gene 2.0 ST Arrays (Affymetrix). The microarrays were scanned on a GeneChip® scanner 3000 (Affymetrix).

### RNA Sequencing

Spatial RNA sequencing was performed using fresh human skin samples (from three separate individuals). Skin samples were incubated with Dispase II (Stemcell Technologies) for 1 h at 37° C. permitting separation of the epidermis. The dermis was separated into papillary (upper 100 mm) reticular (200-500 mm) dermis by microdissection under a dissecting microscope. Separated papillary and reticular dermis samples were subsequently transferred to lysis buffer containing 2-mercaptoethanol (PureLink RNA micro scale kit, Invitrogen) and homogenized for 2 minutes using a mechanical homogenizer (Polytron, Kinematica). Subsequent RNA extraction was performed using the PureLink micro kit according to the manufacturer's instructions. Library preparation was performed according to the SmartSeq2 protocol⁶⁶.

For single cell RNA sequencing, single cells were isolated by flow cytometry (as above) and sorted into individual wells of a 96 well plate containing 2 μl lysis buffer (0.2% (vol/vol) Triton X-100 and 2 U/ul recombinant RNase inhibitor (Clontech). Library preparation was performed with SmartSeq2 followed by the Nextera XT protocol (Illumina). Sequencing was performed on the Ilumina Hiseq2000 or Hiseq 25000 with TruSeq SBS v3 chemistry. Reads were mapped with Tophat2⁶⁷. Gene-specific expression was quantified using featureCounts⁶⁸.

### Graphing and Statistical Analysis

Graphs were generated using Excel and GraphPad Prism 6 software. Data are means±standard error of the mean (SEM). One-way ANOVA parametric test with Bonferroni Post-Test or student's tests were performed for experiments, with P<0.05 considered significant. Statistical analysis and visualization of single cell sequencing data was performed using the Seurat package for R (Satija Lab).

### Results

Differential Expression of Genes Associated with Wnt, ECM and Immune Signalling in Neonatal Mouse Fibroblast Subpopulations

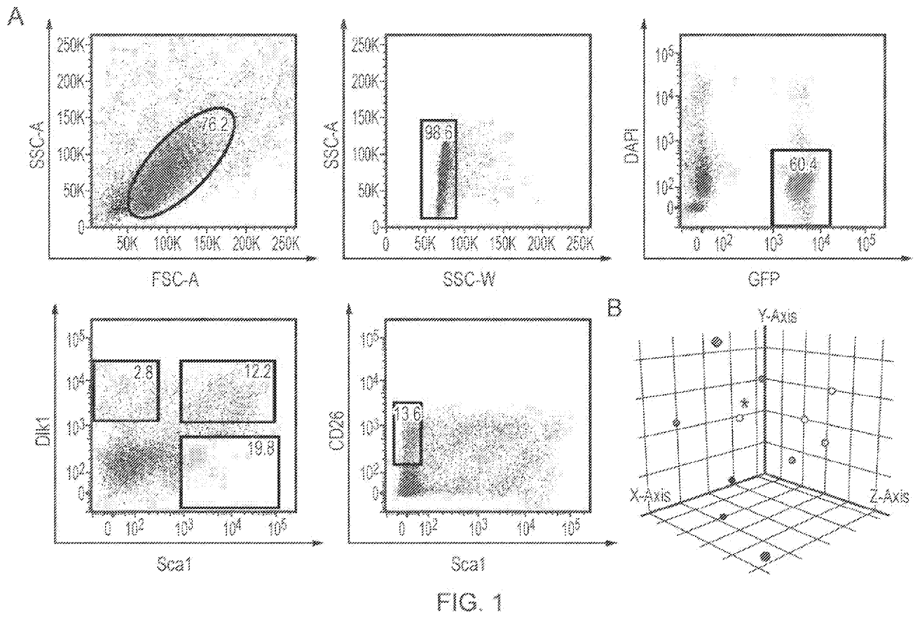

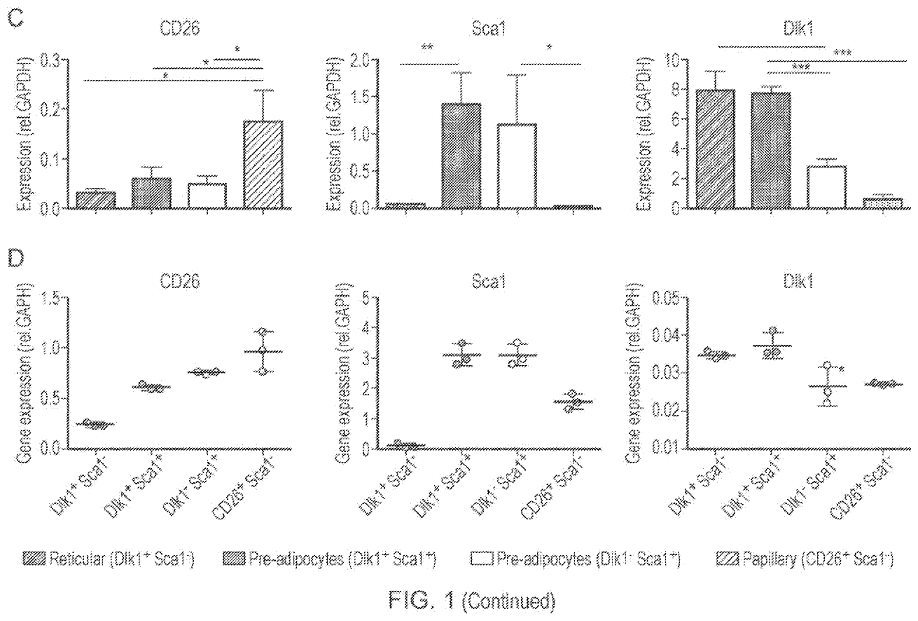

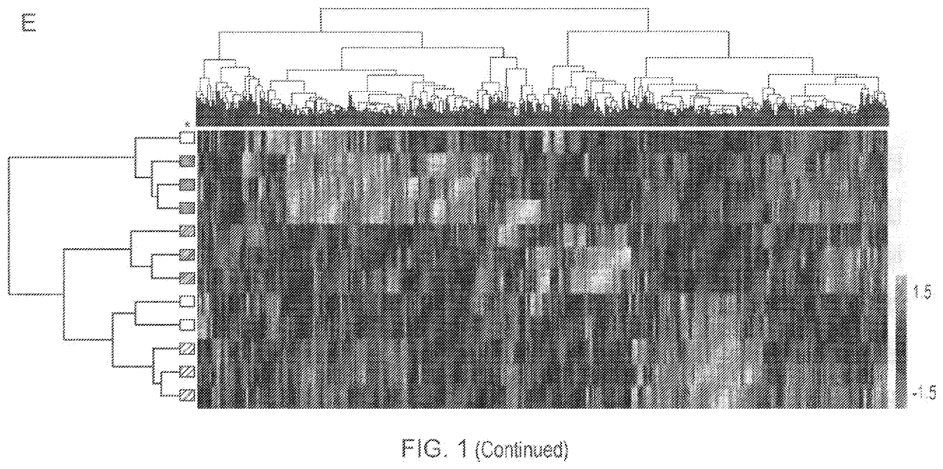

We have previously reported that GFP+ fibroblasts isolated from the back skin of PdgfraHBeGFP reporter mice at postnatal day 2 (P2) can be separated by flow cytometry on the basis of expression of cell surface markers CD26, Sca1 and Dlk1¹³. To identify additional markers and signalling pathways that distinguish these cell populations we performed transcriptomic analysis of flow sorted P2 GFP+ fibroblasts (FIG. 1). Reticular fibroblasts were isolated by gating for Dlk1+Sca1− cells. Two separate pre-adipocyte populations are positive for Sca1 and can be distinguished from one another using Dlk1: Dlk1+Sca1+ cells and Dlk1− Sca1+ cells. Papillary cells were isolated as CD26+Sca1−. 80,000 cells of each population were sorted from three separate mice (FIG. 1A). RNA was extracted and subjected to Affymetrix microarray analysis and qPCR validation of CD26, Sca1 and Dlk1 expression (FIG. 1B-E). Marker expression was confirmed in mRNA isolated from flow sorted populations (FIG. 1C) and in the microarrays (FIG. 1D).

Principal component analysis (PCA) was employed to assess the relationships between the different cell populations (FIG. 1B). The samples aligned along the PC1 (y) axis according to their differentiated state. The two Sca1+(pre-adipocyte) populations were located closest together in PC1. One of the Dlk1−Sca1+ samples clustered with the Dlk1+Sca1+ samples (asterisk, FIG. 1B) and had higher levels of Dlk1 than the other Dlk1−Sca1+ samples in the microarray (asterisk, FIG. 1D), most likely reflecting a technical failure during flow sorting. This sample was therefore excluded from subsequent analysis. Heirarchical clustering of gene expression levels confirmed that, with the exception of the outlier Dlk1−Sca1+ sample (asterisk), the same cell populations from different mice clustered together (FIG. 1E).

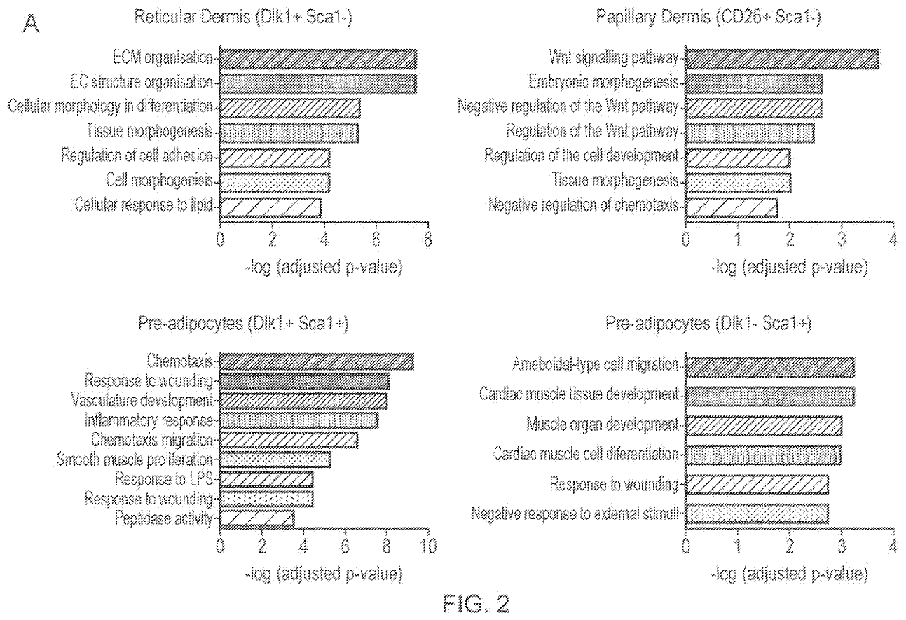

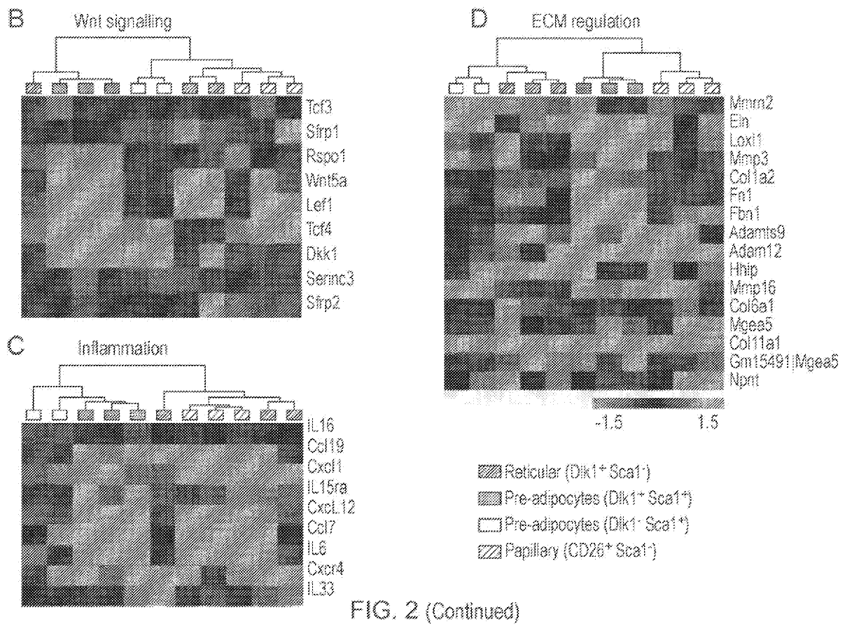

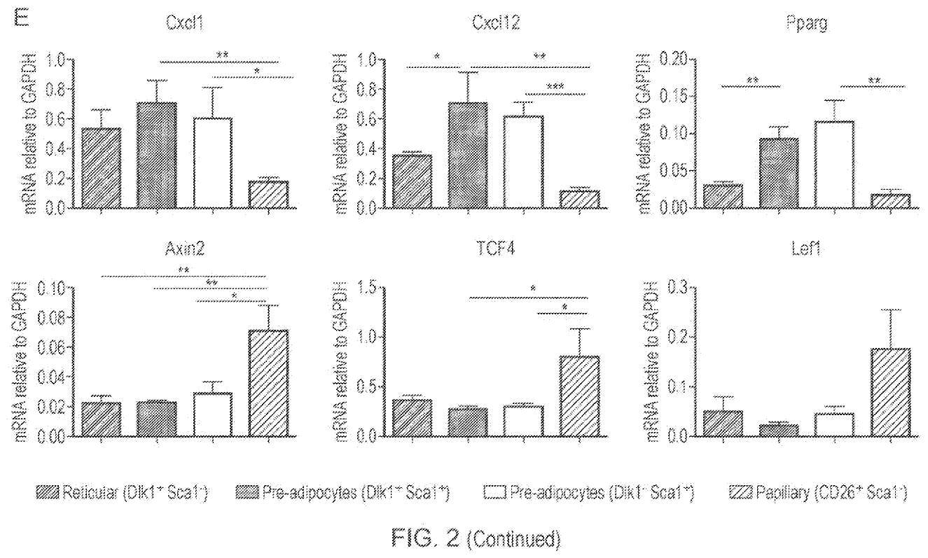

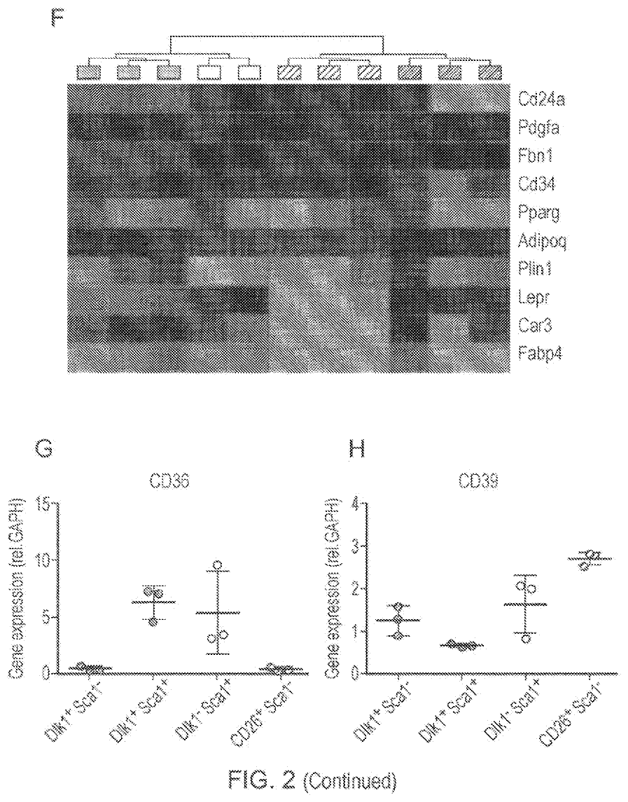

Gene ontology (GO) term analysis of differentially expressed genes (FIG. 2A) confirmed previous observations showing upregulation of the Wnt signaling pathway in the papillary fibroblasts^{13,18}. The top GO term for reticular fibroblasts was ECM organisation, consistent with these cells playing a major role is dermal ECM deposition, while the Dlk1−Sca1+ top GO terms related to muscle, which might reflect the presence of fibro/adipogenic progenitors in the underlying panniculus carnosus muscle¹⁹. The top pathways in Dlk1+Sca1+ cells related to chemotaxis and inflammation. Dlk1+Sca1+ cells expressed higher levels of genes encoding fibrillar ECM proteins, such as fibrillin (FBN1), than Dlk1−Sca1+ cells (FIG. 2E). Genes linked to fibrotic inflammation (e.g. IL-6, CCL7 and CXCL12), were upregulated to a greater extent in Dlk1+Sca1+ cells than Dlk1−Sca1+ cells (FIG. 2B). Heat maps showing examples of differentially expressed Wnt, ECM and inflammation-associated genes are shown in FIG. 2B-D. Differential expression of several of the genes was confirmed by Q-PCR: Wnt pathway genes Tcf4, Lef1 and Axin 2 were more highly expressed in CD26+Sca1− papillary fibroblasts than in the other populations, while Cxcl1 and Cxcl12 were significantly downregulated in papillary fibroblasts (FIG. 2E).

Sca1+ mouse dermal fibroblasts have the capacity to differentiate into adipocytes in vivo and in culture^{13,20}; however, Dlk1+Sca1− fibroblasts also have adipogenic activity¹³. To gain insights into the nature of the two pre-adipocyte populations we compared expression of a panel of adipogenic markers (FIG. 2F). Both Dlk1+Sca1+ and Dlk1−Sca1+ populations expressed higher levels of known pre-adipocyte markers²¹, such as Pparg and Fabp4, than the other populations (FIG. 2F). However, reticular fibroblasts (Dlk1+Sca1−) had the highest levels of the pre-adipocyte marker CD24⁸ (FIG. 2F). Both Sca1+ populations expressed higher levels of the fatty acid transporter CD36²² than the other fibroblast subpopulations (FIG. 2G), while CD39 was selectively upregulated in CD26+Sca1− cells (FIG. 2H).

We conclude that neonatal mouse papillary fibroblasts are distinguished from the other fibroblast populations by having an elevated Wnt gene signature, while Dlk1+Sca1+ cells have elevated expression of ECM and inflammatory genes. Although both Sca1+ populations are capable of differentiating into adipocytes they have distinct gene expression profiles, and cells that express the pre-adipocyte marker CD24 lie within the reticular (Dlk1+Sca1−) population.

### Spatial Transcriptional Profiling of Human Dermis

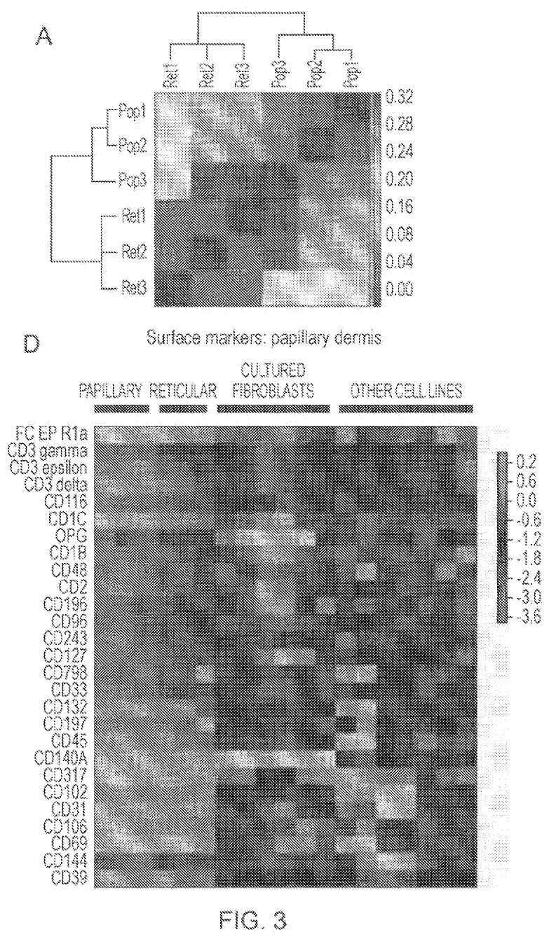

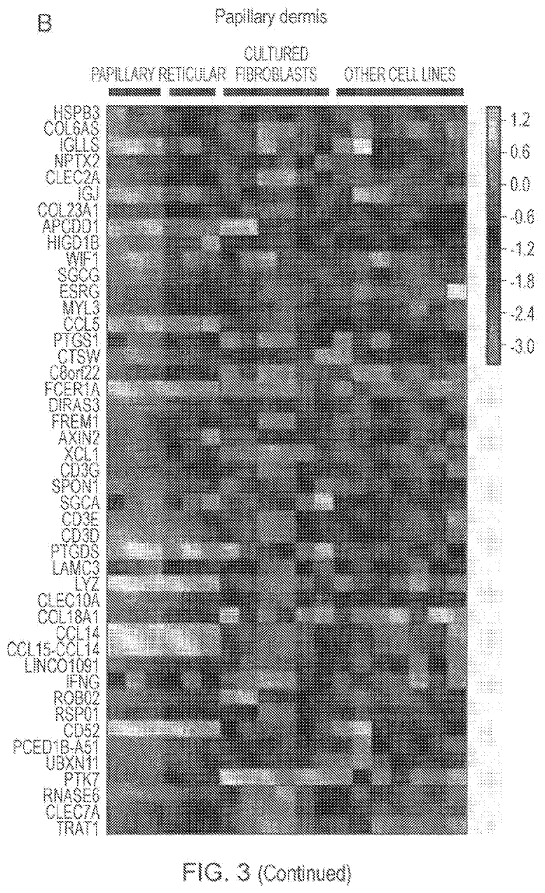

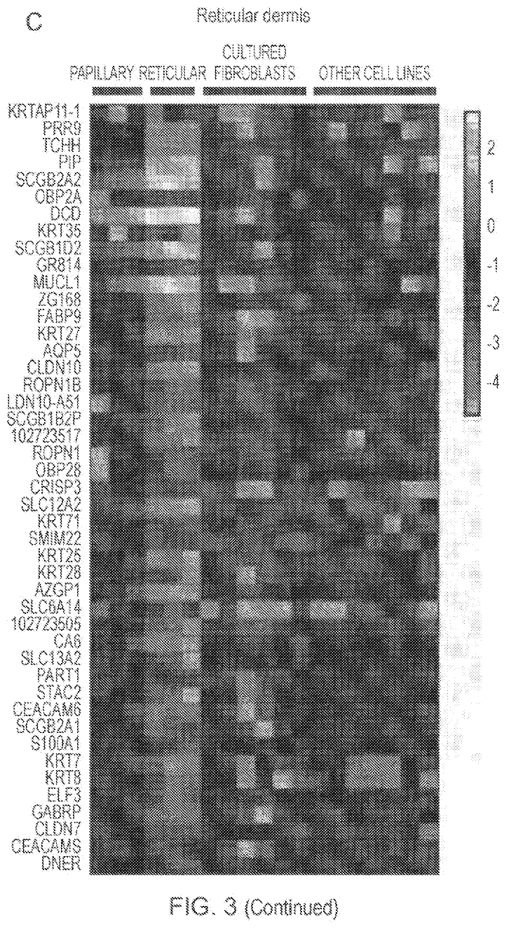

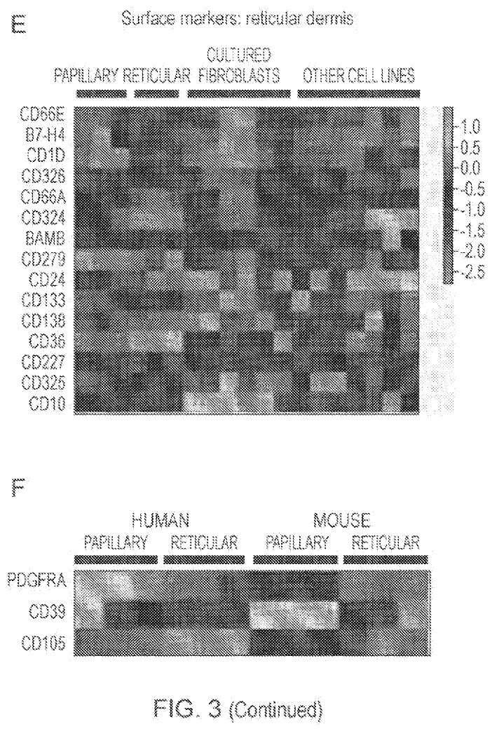

In order to identify markers of human fibroblast subpopulations we hypothesized that spatial differences in gene expression are conserved between mouse and human. To test this, we enzymatically removed the epidermis from human skin samples (three adult female breast samples from separate individuals) and then microdissected papillary from reticular dermis under a dissecting microscope. RNA was extracted separately from the upper 100 mm (‘papillary’) and 200-500 mm (‘reticular’) human dermal layers and subjected to amplification and RNA sequencing. Hierarchical clustering of gene expression revealed that cells in papillary and reticular dermis had distinct gene expression profiles, and that samples from the same spatial location derived from different individuals co-associated (FIG. 3A).

To identify pan-fibroblast markers³, we compared our gene expression profiles from upper and lower human dermis with published RNA-seq data from 6 cultured human fibroblast lines and 7 non-fibroblast cell lines. Pan-fibroblast markers were defined as genes expressed at a high level in both layers of the dermis and all cultured fibroblast lines, but not detectable in other cultured cell types. The only cell surface markers meeting these criteria were the known markers CD90²³ (albeit that CD90 is also expressed in human ES cells), PDGFRα and PDGFRβ². However, the analysis did identify the small leucine-rich proteoglycans lumican (LUM) and decorin (DCN) as secreted pan-fibroblast markers.

Next we analysed the genes that were differentially expressed in the different layers of human dermis to find novel markers for papillary and reticular fibroblasts (FIG. 3B, C). One of the most highly enriched markers in papillary dermis was the α5 chain of collagen VI (COL6A5). Collagen VI is present in most connective tissues where it assembles to form structurally unique microfibrils and is often found in association with basement membranes²⁴⁻²⁶. COL23A1 was also overexpressed in the papillary versus reticular dermis. This analysis additionally identified increased expression of components of the Wnt pathway (WIF1, APCDD1, RSP01, AXIN2) in papillary dermis, suggesting evolutionary conservation of differential Wnt signalling between mouse and human (cf. FIG. 2B). The lower dermis did not have a characteristic ECM signature; however, several members of the secretoglobin superfamily (SCGB2A2, SCGB1D2, SLC12A2) were highly expressed. Hair follicle-specific genes (KRTAP11-1, TCHH) were amongst the most strongly enriched in the reticular dermis, which is likely due to the lower hair follicle remaining embedded within the dermis when the epidermis was removed prior to RNA isolation. High expression of the breast epithelial marker MUCL1 was also a feature of the lower dermis, indicating residual mammary epithelial cells within the preparation.

For functional studies, cell surface markers that distinguish fibroblast subpopulations are very valuable. We therefore filtered the list of differentially expressed genes to identify cell surface markers enriched in papillary (FIG. 3D) and reticular (FIG. 3E) human dermis. Although CD3γ, CD3δ and CD3ε were significantly enriched in papillary dermis this most likely reflected differences in the content of T cells rather than fibroblast subpopulations. We also identified cell surface markers that were differentially expressed in both mouse and human dermal lineages (FIG. 3F). No conserved markers of reticular lineages were identified; however CD39 was identified as a conserved marker of papillary dermal lineages in both mouse and humans.

To validate differential expression of the genes identified in RNA sequencing, we performed antibody labelling on skin sections from three individuals. We confirmed that COL6A5 expression was restricted to papillary dermal fibroblasts (FIG. 4A, B). This pattern of expression has been described in two independent reports^{27,28} and thus COL6A5 is a robust marker for papillary dermal fibroblasts. Immunostaining for APCDD1 (FIG. 4C, D) HSPB3 (FIG. 4E, F) and WIF1 (FIG. 4G, H) confirmed differential expression of these markers in papillary dermis (FIG. 3B). Consistent with their expression in mouse fibroblast subpopulations (FIG. 2G, H), CD36 was preferentially expressed in the lower reticular dermis and hypodermis (FIG. 4I, J) and CD39 in the papillary dermis (FIG. 4K, L).

We conclude that in neonatal mouse and adult human dermis there is differential gene expression in fibroblasts of the reticular and papillary dermis, that differential Wnt signalling is an evolutionarily conserved trait, and that CD36 and CD39 are surface markers for prospective isolation of different fibroblast subpopulations.

### Functional Heterogeneity of Flow Sorted Human Fibroblasts

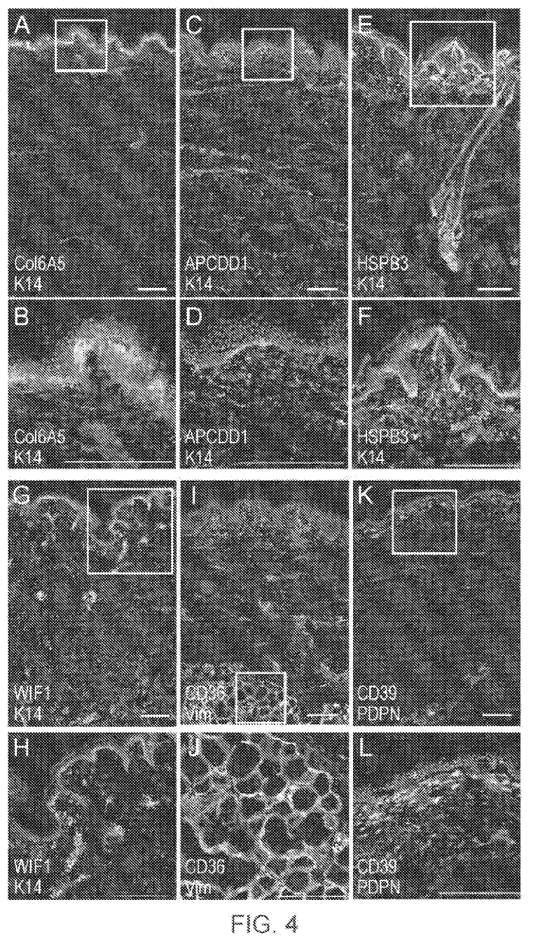

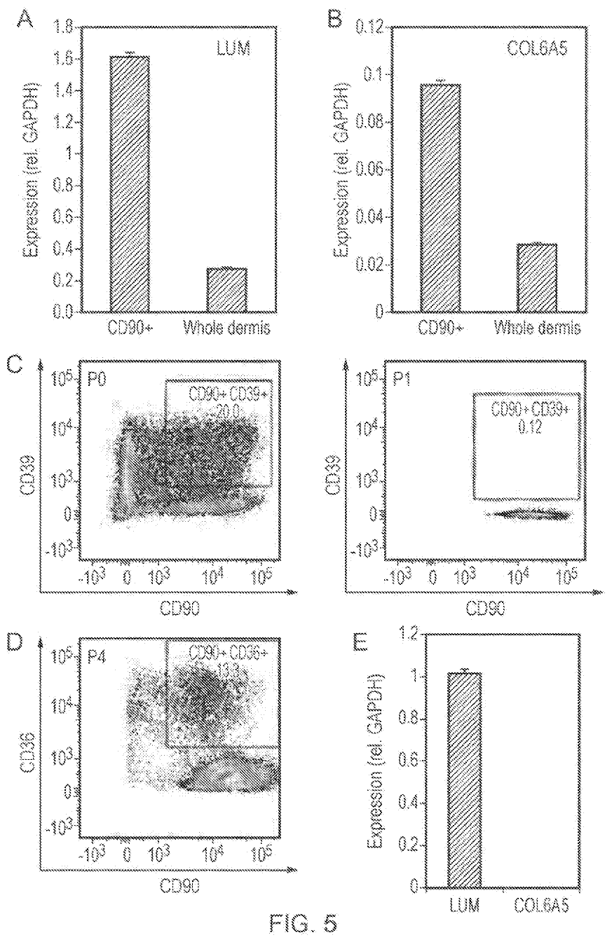

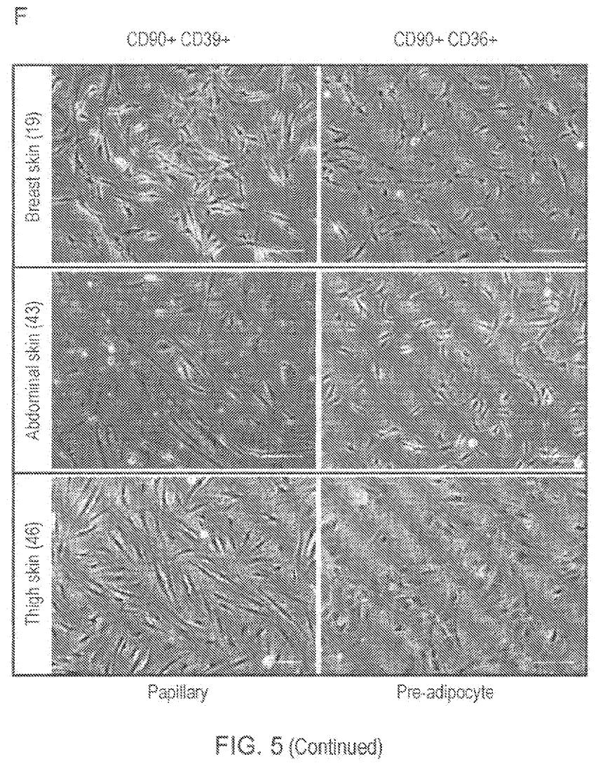

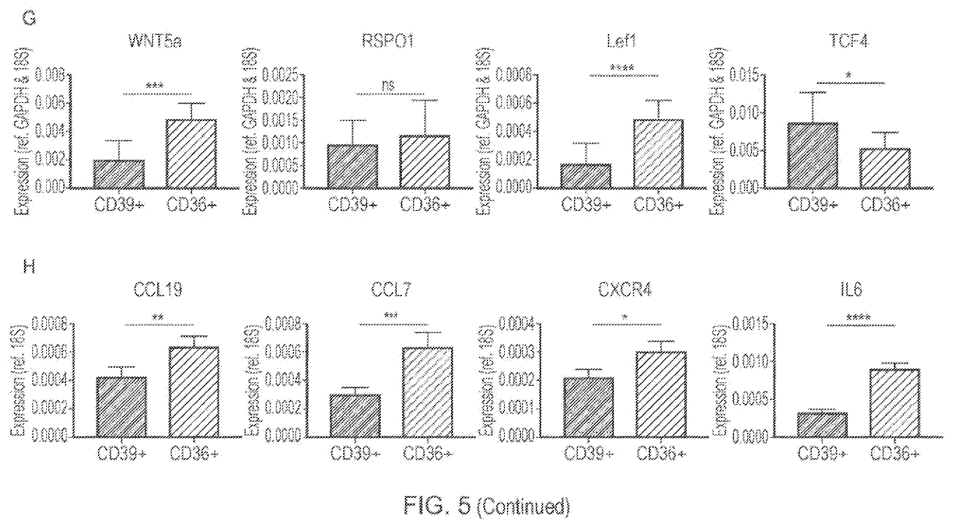

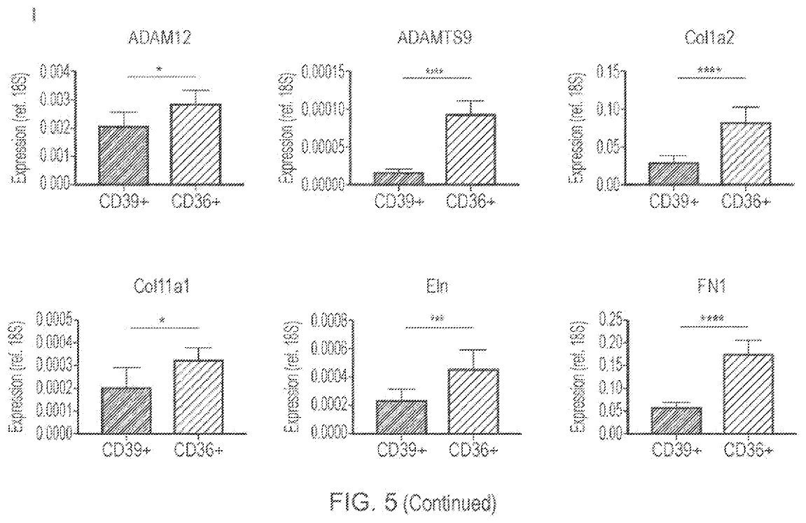

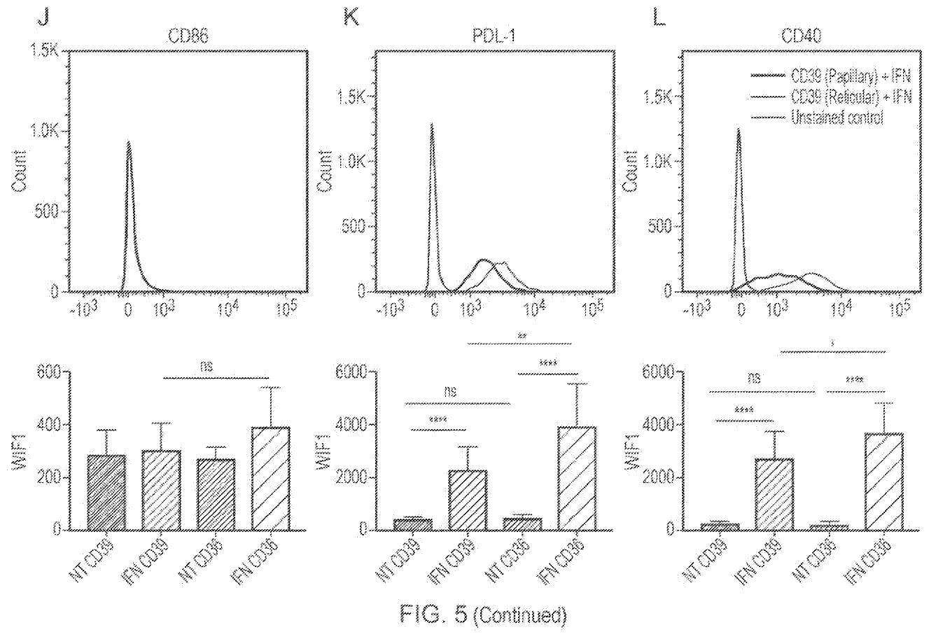

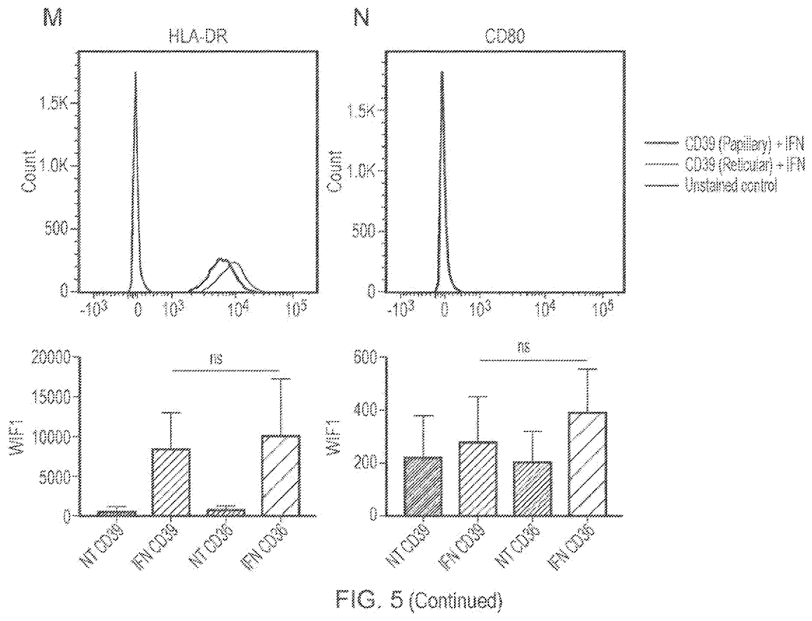

For therapeutic applications ex vivo expansion of specific dermal fibroblast subpopulations would be desirable if the cells retained distinct characteristics in culture. Based on our analysis of mouse and human fibroblasts we therefore flow sorted human fibroblasts that were lin−(i.e. CD31−CD45−E-cadherin-)CD90+CD39+(papillary) or lin−CD90+CD36+(lower reticular/hypodermal) and compared their properties following expansion in culture for up to 4 passages (FIG. 5). We confirmed that expression of LUM and COL6A5 was enriched in CD90+ fibroblasts relative to total dermis (FIG. 5A, B). After a single passage, the expression of CD39 and COL6A5 was completely lost; however, expression of CD90, LUM and CD36 was maintained (FIG. 5C-E). This demonstrates that culture itself leads to the loss of fibroblast markers rather than cellular competition.

Although expression of CD39 and COL6A5 was rapidly lost in culture, lin− CD90+CD39+ and lin−CD90+CD36+ populations exhibited differences in morphology in culture. In some samples CD39+ cells exhibited a spindle morphology while CD36+ cells had a more epithelioid shape (FIG. 5F). However, these differences were not consistent between tissue samples, potentially reflecting the isolation of cells from skin of different ages and body sites (FIG. 5F). qPCR revealed that expression of genes encoding several ECM components (FIG. 5I) and inflammatory mediators (FIG. 5H) continued to be more highly expressed in CD36+ cells. However, the elevated expression of Wnt pathway genes associated with papillary dermis was lost (FIG. 5G).

To examine the functional significance of differential expression of inflammatory mediators (FIG. 5H) we performed interferon stimulation assays (FIG. 5J-N). We observed clear differences in the response of lin−CD90+CD39+(upper dermis) in comparison to lin−CD90+CD36+(hypodermis) with a significant reduction in the upregulation of PDL-1 and CD40 in CD39+ cells. This is consistent with an anti-inflammatory phenotype for upper dermal fibroblasts.

Mouse studies have identified a role for reciprocal Wnt signalling between basal keratinocytes and upper dermal fibroblasts in the regulation and maintenance of the epidermal stem cell compartment^{18,29-31}. Although the papillary Wnt gene signature was lost in culture (FIG. 5G), it has been previously demonstrated that cultured fibroblasts derived from the papillary dermis support the formation of a normal stratified epidermis in three dimensional organotypic culture more effectively than fibroblasts from the reticular dermis¹⁵⁻¹⁷. We therefore employed this as a functional assay to assess the ability of cultured fibroblast subpopulations to repopulate decellularised human dermis (DED) and support the formation of an architecturally normal epidermis. Flow sorted lin−CD90+CD39+, lin−CD90+CD39− and lin−CD90+CD36+ cells were expanded in culture and then introduced into the upper surface of DED. Subsequently, primary human keratinocytes were added to the surface of the dermis and cultured at the air-liquid interface.

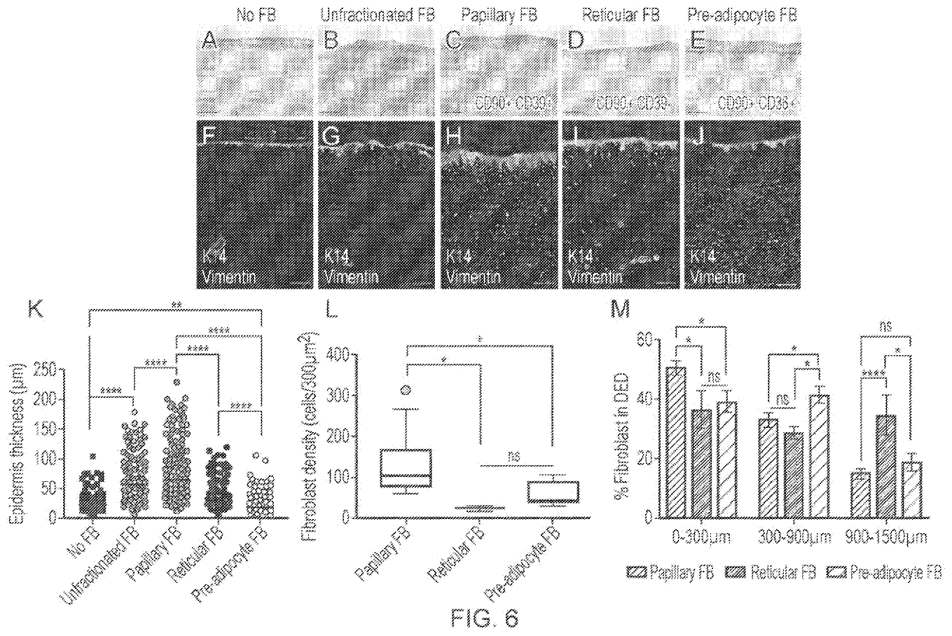

After 3 weeks in culture, in the absence of fibroblasts, the epidermis had fewer viable cell layers than normal epidermis and lacked the undulations, known as rete ridges, that are characteristic of the healthy tissue (FIG. 6A, F). The addition of unfractionated fibroblasts (CD90+) led to the formation of a thicker epidermis with some rete ridges (FIG. 6B, G). When fibroblast subpopulations were prospectively isolated, lin−CD90+CD39+ cells more effectively supported the formation of a multi-layered epithelium (FIG. 6C, H, K) than lin−CD90+CD39− cells (FIG. 6D, I) and lin−CD90+CD36+ cells (FIG. 6E, J). After seeding, lin−CD90+CD39+ remained largely restricted to the upper dermis, while lin−CD90+CD39− and lin−CD90+CD36+ cells extended to mid and deep dermis (FIG. 6L, M) and fibroblast density was highest in DEDs reconstituted with lin−CD90+CD39+ cells (FIG. 6L).

In summary, our functional analysis demonstrates that, not withstanding changes in gene expression associated with culture, prospectively isolated fibroblast subpopulations retain differences in immunological function and the ability to support the formation of an architecturally normal epidermis in 3-dimensional organotypic cell culture.

Single Cell Transcriptional Profiling of Human Dermal Fibroblasts Identifies Subpopulations that are not Spatial Segregated

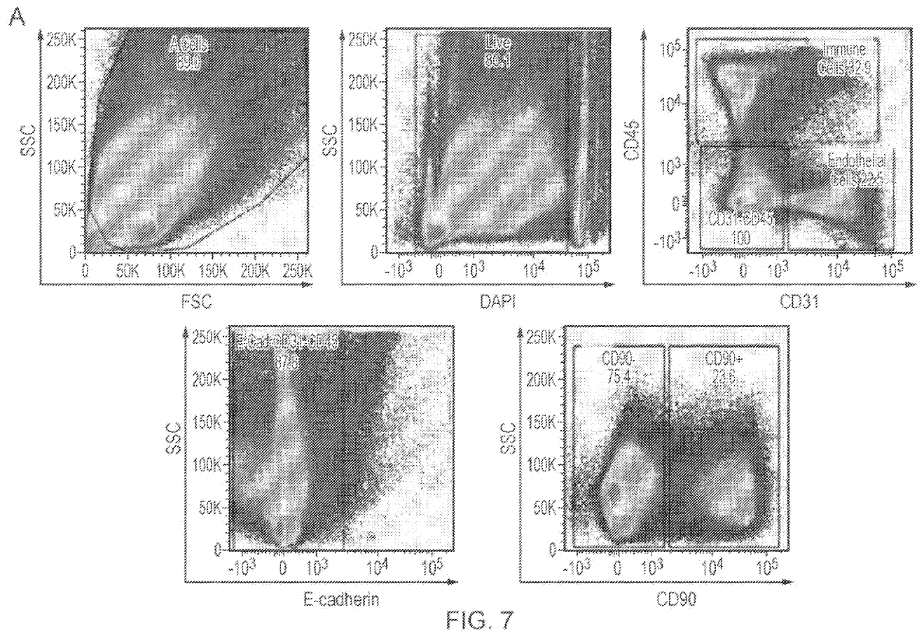

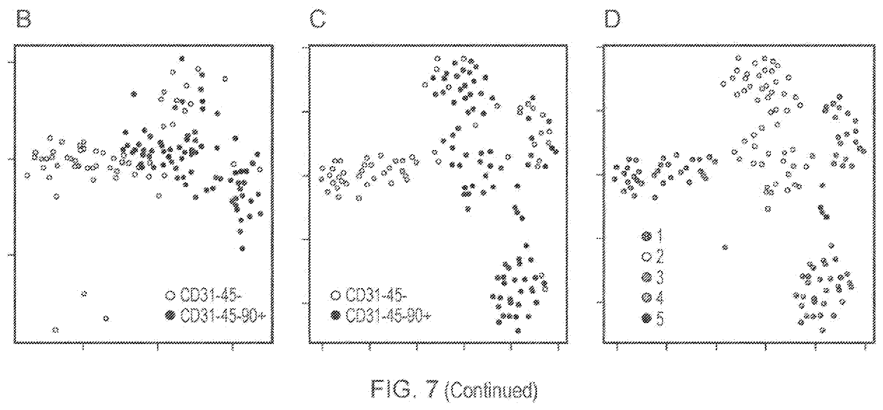

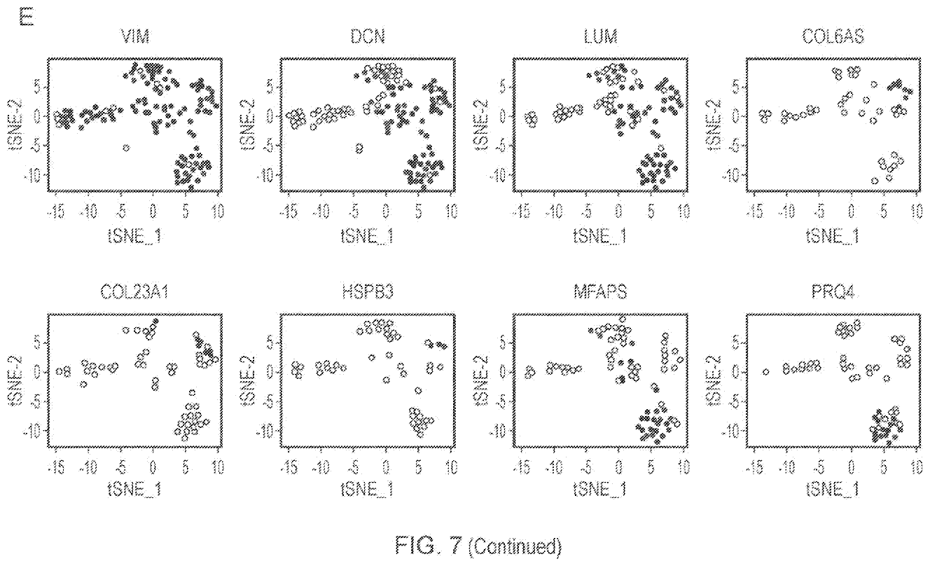

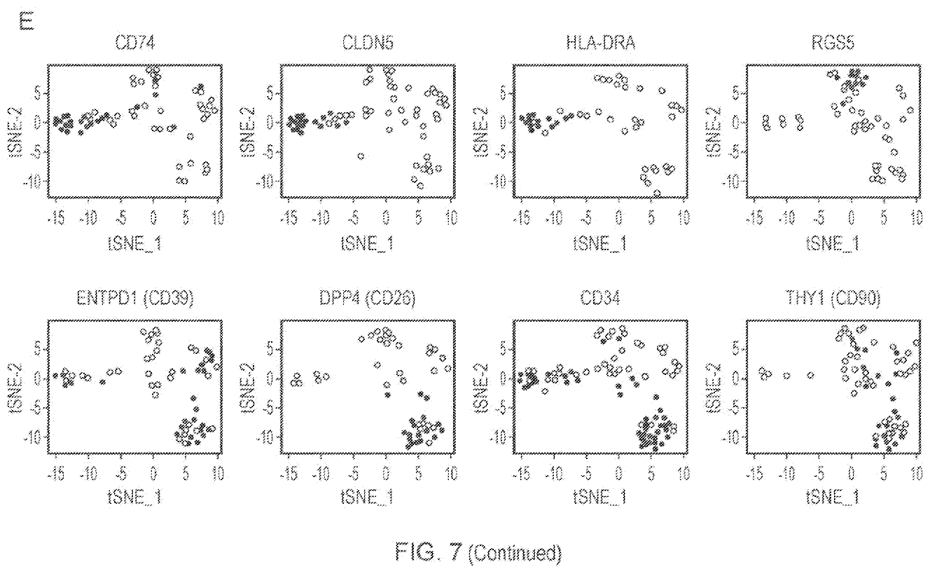

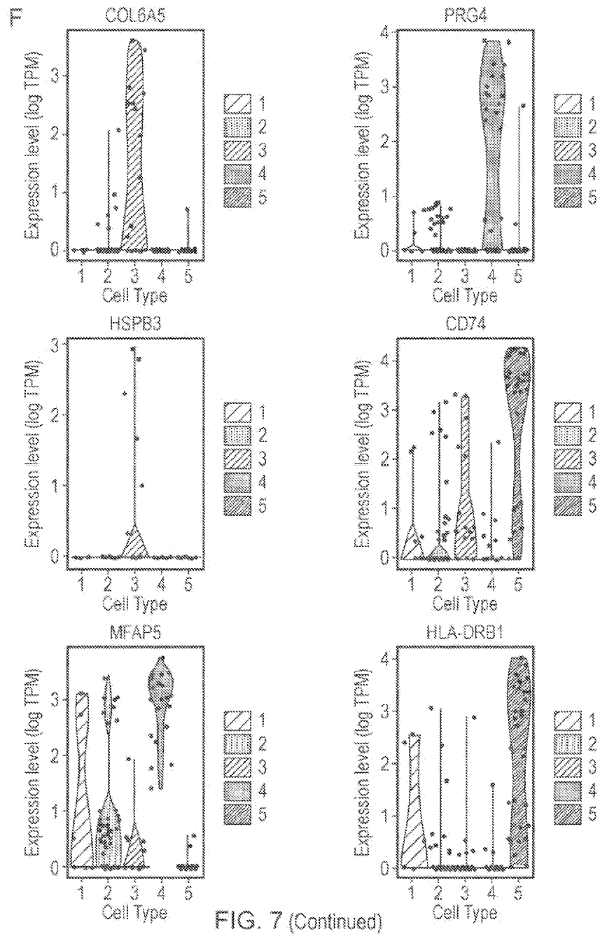

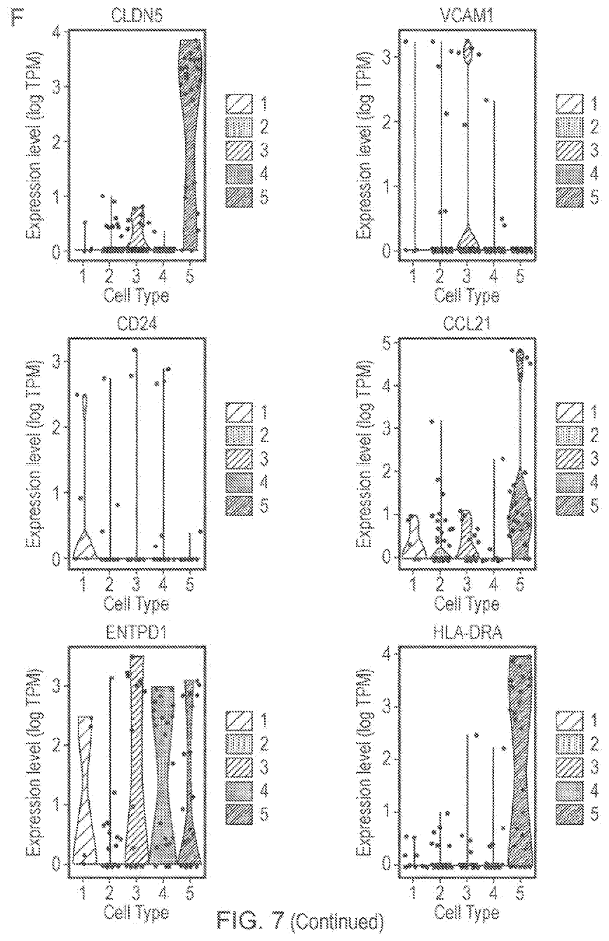

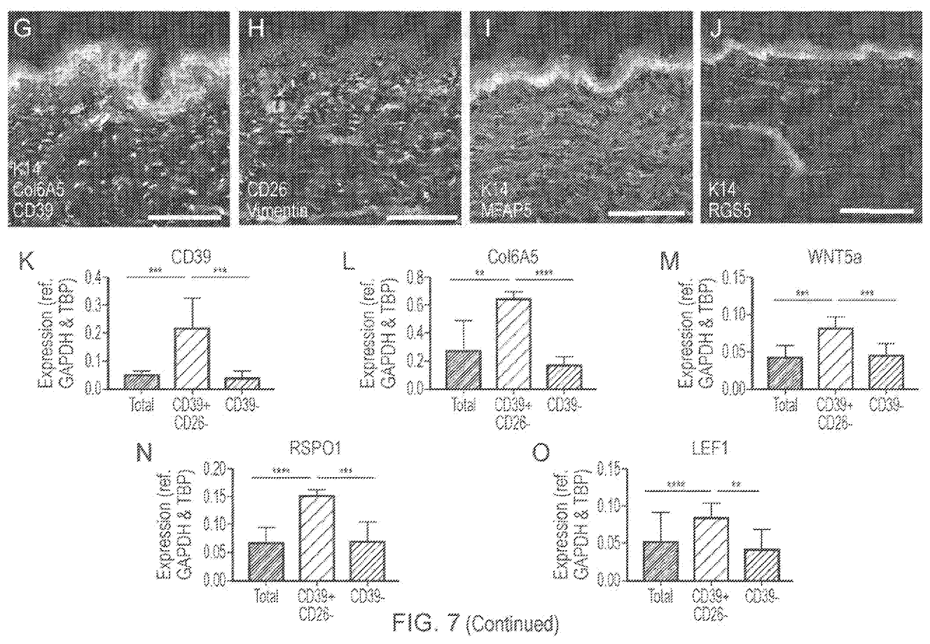

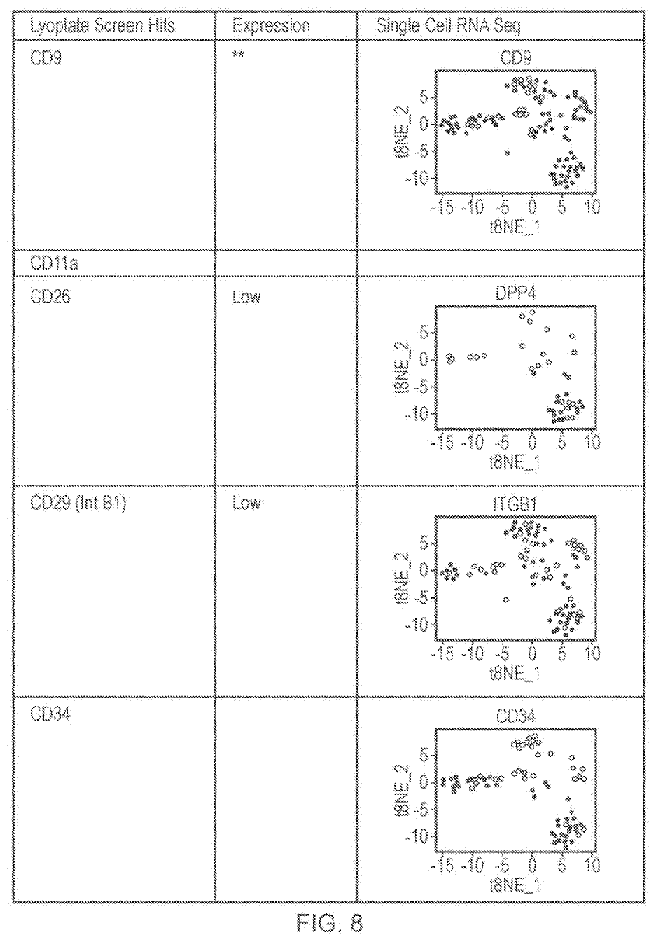

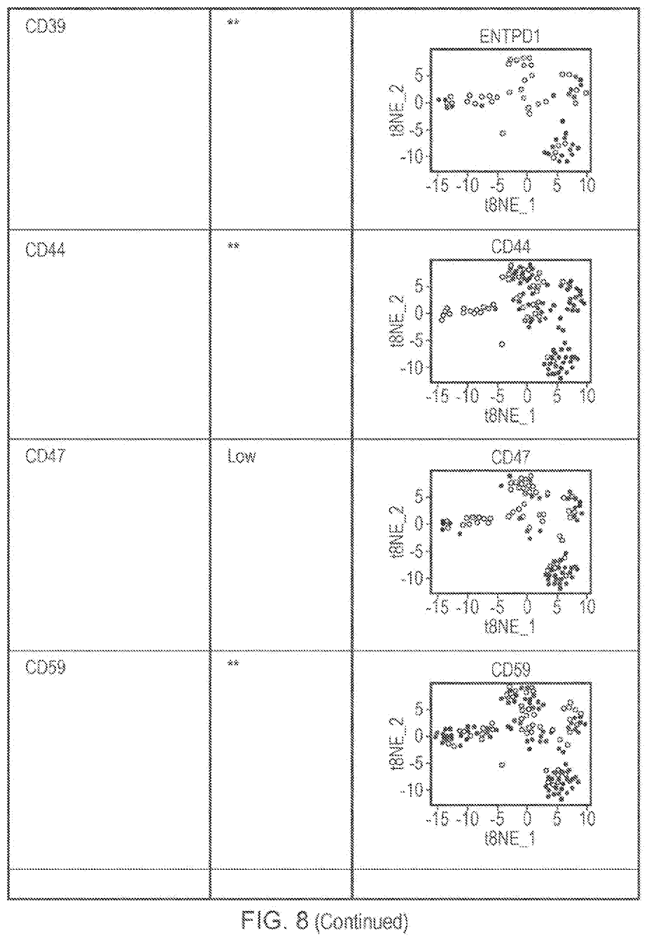

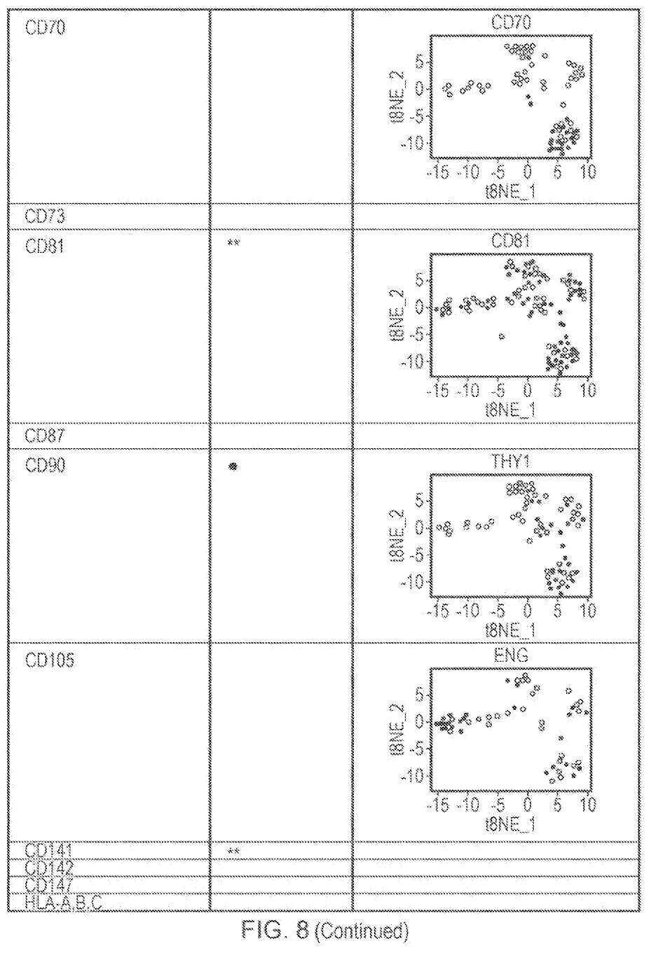

Although our analysis of fibroblasts in different layers of human dermis supports the concept that fibroblasts are heterogeneous, it is only able to detect fibroblast subpopulations that are spatially segregated between papillary and reticular dermis. Therefore, in order to undertake a more comprehensive assessment of fibroblast subpopulations we isolated both lineage lin− and lin−CD90+ cells from human dermis after enzymatic removal of the epidermis (FIG. 7A). For 5 samples of primary human dermis, 12.2% (S.D. 6.7%) of cells were CD90+, 9.4% (S.D. 4.7%) were lin−CD90+ and 39.6% (S.D. 16.4%) were lin−CD90−. Thus fibroblasts constitute a small proportion of dermal cells, the majority being CD31+ endothelial cells (8.1% S.D. 5.2%), CD45+ (11.6% S.D. 7.4%) haemopoietic cells and other cell types including sweat glands, neuronal cells and keratinocytes from hair follicles.

To define additional fibroblast subpopulations, we performed single cell RNA sequencing for a total of 184 cells. In order to enrich for fibroblasts half of the cells were flow sorted lin−CD90+ cells. However, since we did not wish to exclude potentially novel fibroblast subsets that were CD90−, half of the cells were lin−CD90+/−. tSNE analysis (FIG. 7B-D) was performed on global patterns of gene expression for each individual cell; this method groups cells with similar gene expression. Automated clustering of the tSNE analysis (FIG. 7D) identified 5 groups of cells, although Group 1 (red) contained only 5 cells.

To characterise the fibroblast subpopulations, we computationally identified genes that were significantly overexpressed in each group. Vimentin, a mesenchymal cell marker, was expressed in all groups, establishing that no epidermal cells were profiled (FIG. 7E, F). Decorin (DCN) and lumican (LUM)—the pan-fibroblast markers we identified by spatial transcriptomics (FIG. 3)—were expressed in all groups with the exception of Group 5. Group 5 also contained no lin−CD90+ cells. COL6A5, COL23A1 and HSPB3, identified by spatial transcriptional profiling as localized to the papillary dermis (FIG. 3B), were highly enriched in Group 3, suggesting that this group represents papillary fibroblasts.

CD74 and HLA-DR4− both components of MHC class II³²—and CLDN5, a component of tight junctions³³, marked Group 5. Although these cells were vimentin+ and lin− the lack of DCN and LUM and expression of CD74 (macrophage inhibitory factor receptor) and HLA-DR indicate characteristics of macrophages/dendritic cells^{32,34}. However, CLDN5, another marker of Group 5 cells, is an endothelial marker³⁵. Interestingly expression of Pparg (PPARG) and to a lesser extent CD36, pre-adipocyte markers, was also enriched in Group 5, suggesting that at least a fraction of pre-adipocytes are CD90−. Furthermore a subpopulation of group 5 cells express TEK (TIE2) and might correspond to TIE2-expressing monocyte/macrophages³⁶.

Although Group 2 was one of the largest cell clusters, there were few specific markers. One of those, RGS5, is well characterized as a marker of pericytes³⁷ and antibodies to RGS5 labelled blood vessel-associated pericytes throughout the dermis (FIG. 7J). RGS5-positive cells were limited to the upper half of Group 2, suggesting that cells in the lower half of Group 2 represents an alternative cellular identity.

CD26, MFAP5 and PRG4 were identified as markers of Group 4. CD26 was also a marker of Group 1 cells, while MFAP5 was expressed by cells in Group 2. Antibody labelling indicated that, in contrast to neonatal mouse dermis (FIG. 1), cells expressing CD26 were absent from adult papillary human dermis (FIG. 7H). MFAP5 was expressed throughout the dermis (FIG. 5I). By flow sorting papillary cells on the basis that they were CD39+CD26− (FIG. 7G, H), we could enrich for expression of the papillary markers COL6A5, WNT5A, RSPO1 and LEF1 (FIG. 7K-O).

We conclude that fibroblast identity is not restricted by spatial compartmentalization within the dermis, except in the case of papillary fibroblasts and that by single cell RNA sequencing we can identify additional fibroblast subpopulations.

### DISCUSSION

Our results support the emerging concept^{13,14} that rather than a single homogenous cell type, fibroblasts represent a family of related cell types with specialized functions in the synthesis and maintenance of extracellular matrix and the coordination and regulation of neighbouring cell types. Through a combination of spatial and single-cell transcriptional profiling, we have identified at least four fibroblast subpopulations within the human dermis. The first of these, which has a cell surface phenotype of lin−CD90+CD39+CD26− is characterized by the expression of specific collagen chains such as COL6A5 and is localized to the upper dermis. The second is lin−CD90+CD39+CD26+ and is located throughout the remainder of the dermis. The remaining groups are lin−CD90+CD39−RGS5+ cells that correspond to pericytes³⁷; lin−CD36+ cells which are situated in the lower dermis and represent pre-adipocytes; and lin−CD90+CD39−RGS5− cells which are an as yet uncharacterized fibroblast subpopulation.

In contrast to previous studies of the mouse dermis³⁸⁻⁴¹, dermal papilla associated fibroblasts were not prominent in our dataset, likely reflecting the significantly lower density of hair follicles in human breast skin in comparison to mouse skin. Many of the cell surface markers identified in our previous study of the developing mouse dermis were not conserved in the human. This lack of conservation is not unexpected since there is poor conservation of cell surface markers for haematopoietic stem cells between humans and mice⁴². However interestingly all three studies of dermal fibroblast identity have identified CD26 as a key lineage marker, although the subpopulations marked appear to differ: During early stages of mouse development, CD26 marks an upper dermal lineage; in the adult mouse, CD26 expression is present in a large fraction of the dermis¹⁴. Here we have demonstrated that CD26 expression, whilst present in a large fraction of the human adult dermis is excluded from the uppermost (papillary) fibroblasts. Of note, CD26 has been implicated as a regulator of the inflammatory response⁴³ and CD26+ fibroblasts are reported to contribute to skin fibrosis⁴⁴.

It is likely that the specification of fibroblast cellular identity reflects a combination of both extrinsic signals emanating from the spatial context of the cell, including cell-cell contacts and diffusible cell signalling mediators, and intrinsic mechanisms, including transcriptional and epigenetic regulatory networks. With regards to the former, our results support a key role for signalling via the Wnt pathway in the regulation of dermal fibroblast subpopulation identity. We have found upregulation of Wnt signalling in both upper dermal mouse and human fibroblast lineages. Wnt signalling is required for normal development of mouse⁴⁵ and human dermis⁴⁶ and studies have revealed a central role for Wnt signalling in hair follicle development and regeneration^{47,48,40,49}. Epidermal Wnt signalling is also implicated in the regulation of the hypodermal adipocyte layer^{8,50} and is critical to maintenance of the epidermis⁵¹. We propose that there is a mutually synergistic Wnt-mediated cross-talk between papillary dermal fibroblasts and basal keratinocytes that is responsible for the maintenance of the cellular identity of the papillary dermal fibroblasts. Loss of this signalling in culture may explain the rapid loss of papillary cell markers.

In addition to differential expression of Wnt pathway components we observed differential expression of ECM and immunoregulatory genes in subpopulations of mouse and human fibroblasts on isolation from the dermis. We also observed conservation of cell function following in vitro expansion of upper and lower dermal fibroblasts, as evidenced by differences in collagen chain expression, the immunological response to interferon stimulation and ability to support the normal development of a stratified squamous epithelium in 3D-reconstituted organotypic cell culture. Interestingly, whilst differences in fibroblast function are conserved in culture, at least for early passages, the expression of key subpopulation markers was rapidly lost. This finding, in combination with the observation that fibroblasts constitute a small fraction of the cells present in the skin, has implications for studies seeking to understand disease processes on the basis of experiments conducted with cultured fibroblasts and an analogy may be drawn with previous studies that have shown that properties of epidermal stem cells displayed in culture or in transplantation experiments differ from those exhibited in vivo^{1,52}.

The emerging concept that mammalian tissues, such as the dermis, contain fibroblasts with differing functional identities has profound implications for the understanding of a wide range of pathological states. Wound healing in the adult is characterized by the formation of an ECM-rich scar lacking normal cutaneous appendages such as hair and sweat glands⁵³. Lineage tracing in the mouse demonstrates that the initial wave of fibroblast migration is derived from lower (reticular) lineage fibroblasts expressing alpha smooth muscle actin¹³. It will be of great interest to determine whether this is conserved in humans, offering potential strategies for the modulation of normal wound healing and the understanding of disease states such as keloidal scaring^{54,55}, scleroderma, and graft versus host disease. Alterations in the quantity or function of fibroblasts subpopulations may also play a role in the characteristic age-associated changes in skin architecture including dermal thinning and flattening of the dermal-epidermal junction with loss of the normal rete ridges^{56,57}.

An understanding of the differential contribution of fibroblast subpopulations to human disease may offer novel strategies for therapy. In this context it could be envisaged that the action of deleterious fibroblast subpopulations could be inhibited, perhaps via inhibition of signalling pathways specific to these subpopulations or via monoclonal antibody-mediated ablation. In support of this concept, experimentally reducing the number of fibroblasts during wound healing in the mouse can reduce the degree of fibrosis⁵⁸. Alternatively, beneficial subpopulations may be expanded ex vivo, perhaps via manipulation of cell signalling pathways. Trials of fibroblast cell therapy are already underway for the treatment of poorly healing ulcers⁵⁹ and epidermolysis bullosa⁶⁰. Of note, papillary fibroblasts appear to be more effective in the construction of tissue-engineered skin substitutes⁶¹.

In summary, through a combination of spatial and single cell transcriptional profiling we have shown that, consistent with previous studies of the mouse dermis, fibroblast subpopulations can be identified within the human dermis. We have identified cell surface markers permitting the prospective isolation of these subpopulations and demonstrated that they exhibit functional specialization. These findings have important implications for the understanding and treatment of diseases characterized by fibroblast dysfunction.

### Example 2

### Methods

### Fibroblast Isolation

Primary fibroblasts were isolated as described above. Different subpopulations of cells were isolated by flow cytometry following depletion of lineage negative (CD45−, CD31−, e-Cadherin−) cells. Fibroblasts were then positively sorted for CD90+, followed by their respective cell surface markers.

### Wound Healing Ex-Vivo Explants

Adult surgical waste skin from consenting patients undergoing plastic surgery was obtained from St George's University Hospitals NHS Foundation Trust, and the study was ethically approved by the National Research Ethics Service Committee UK (HTA Licence No: 12121, REC-No: 14/NS/1073). Excess adipose tissue was dissected off and the skin sterilised by immersing in 10% iodine antiseptic solution, followed by two changes of 70% alcohol and finally in sterile phosphate buffered saline (PBS, Gibco). The skin was cut into 1 cm² pieces and a partial thickness wound created in the centre of each on using a 4 mm punch biopsy. Before fibroblasts were seeded into wounds, the tissue was placed into 6-well hanging cell culture inserts and equilibrated with DMEM, with an air-liquid interface. Primary fibroblast subpopulations (20,000 cells) from another donor were resuspended in 10 μl of DMEM and pipetted into the wound. Explants were incubated at 37° C. in a humidified atmosphere with 5% CO₂ for 2 weeks and then embedded in optimal cutting temperature compound (OCT, Life Technologies) prior to sectioning.

### De-Epidermised Dermis (DED) Organotypic Culture

De-epidermised dermis (DED) was prepared as described previously (Rikimaru et al, Experimental dermatology 6, 214-221 (1997)). Briefly, normal adult human skin or excised keloid scars were cut into 1-2 cm², heated at 52° C. for 20 min and the epidermis separated from the dermis with forceps. The dermis was depleted of cells by 10 freeze-thaw cycles and irradiated once with 60 Gy. Before fibroblasts were seeded into DEDs, the tissue was placed into 6-well hanging cell culture inserts (Millipore) and equilibrated with DMEM. Different primary fibroblast subpopulations (5×10⁵ cells) were injected into each DED using U-100 insulin syringes (BD) from the epidermis surface. The DEDs were then incubated for 24 h completely submerged in DMEM at 37° C. in a humidified atmosphere with 5% CO₂. Medium was changed to FAD medium with an air-liquid interface, and 1×10⁶ keratinocytes were seeded on top of the DED. DEDs were maintained in culture with FAD medium at an air-liquid interface for 3 weeks with media changes every 48 h. Samples were embedded in OCT prior to sectioning.

### Collagen Analysis

For collagen quantification, 16 μm cryosections were stained with Picrosirius red using a standard method (Lattouf et al, J Histochem Cytochem. 2014; 62(10):751-8). Briefly, the sections were fixed with 4% paraformaldehyde/PBS (10 min at room temperature), washed twice with water and stained for 1 h in Picrosirius red solution [0.1% Sirius red F3B (Sigma) in a saturated aqueous solution of picric acid]. After staining, sections were washed twice with acidified water (0.5% acetic acid), dehydrated, cleared with xylene and mounted with DPX mounting medium (Sigma). The images were taken with a Zeiss Axiophot microscope and AxioCam HRc camera under plane polarised light that shows the collagen fibres as green, orange and yellow against a black back-ground. The intensity of light was adjusted to give a linear response for quantification. The quantification of total collagen fibres was performed using Fiji imaging software. The collagen pixels were selected with the colour threshold tool (hue 0-100, saturation 0-255 and brightness 230-255), and the binary images were created and measured based on the selection.

For collagen hybridising peptide (CHP) staining (Hwang et al, 2017; Li & Yu, 2013), 16 μm cryosections were fixed with 4% paraformaldehyde/PBS (10 min at room temperature), permeabilised with 0.1% Triton X-100/PBS (10 min at room temperature), blocked with 5% BSA/PBS (1 h at room temperature) and stained with the indicated primary antibodies and 5 μM B-CHP (BIO300, 3Helix) overnight at 4° C. According to the manufacturer's instructions, the B-CHP probe was heated for 5 min at 80° C. before adding it to the primary antibody mix, which was immediately applied to the tissue sections. Sections were washed three times with PBS and incubated with appropriate secondary antibody and streptavidin-AlexaFluor647 (S32357, Thermo Fisher) for 1 h at room temperature. After washing the sections with PBS and incubating them with DAPI (1 μg/ml diluted 1:50,000) for 10 min at room temperature, the samples were mounted with ProLong® Gold Antifade Mountant (Thermo Fisher). Confocal microscopy was performed with a Nikon A1 confocal microscope using the 20× objective.

### Results

Wounded Skin Seeded with Different Fibroblast Subpopulations, Cultured Ex Vivo for 14 Days

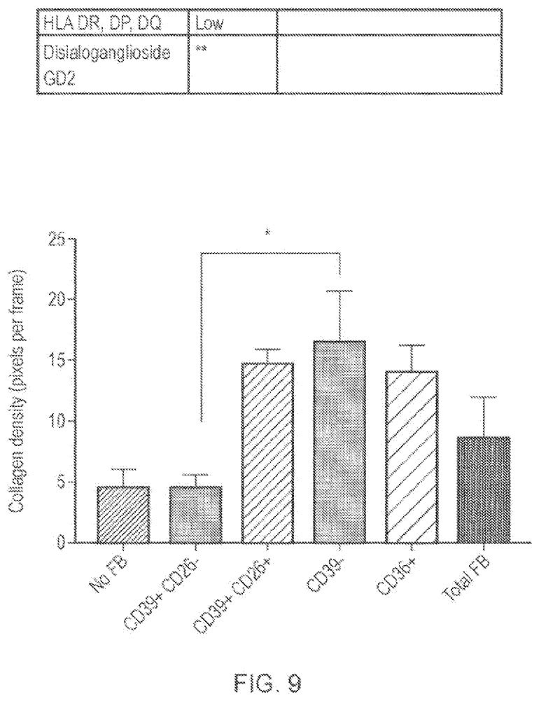

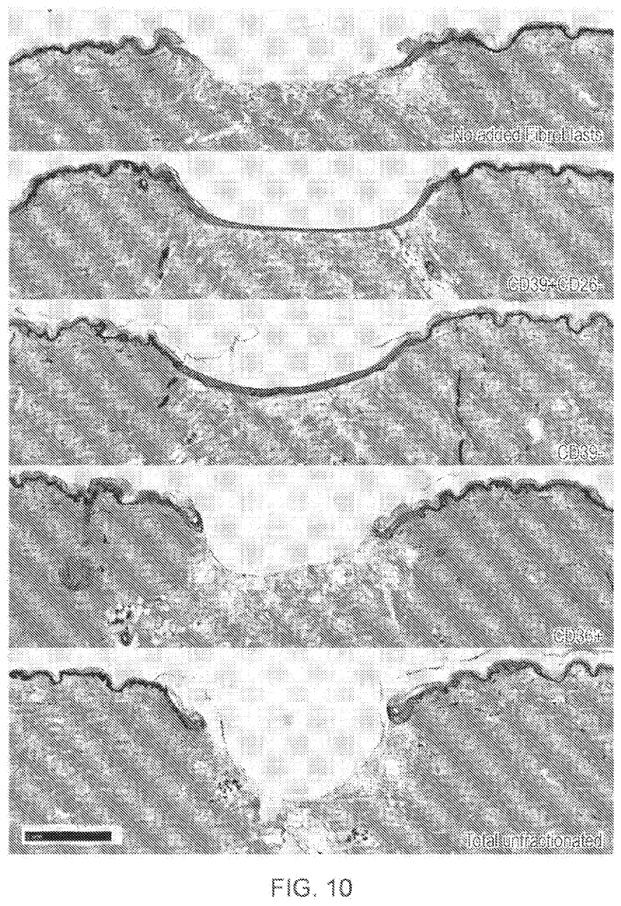

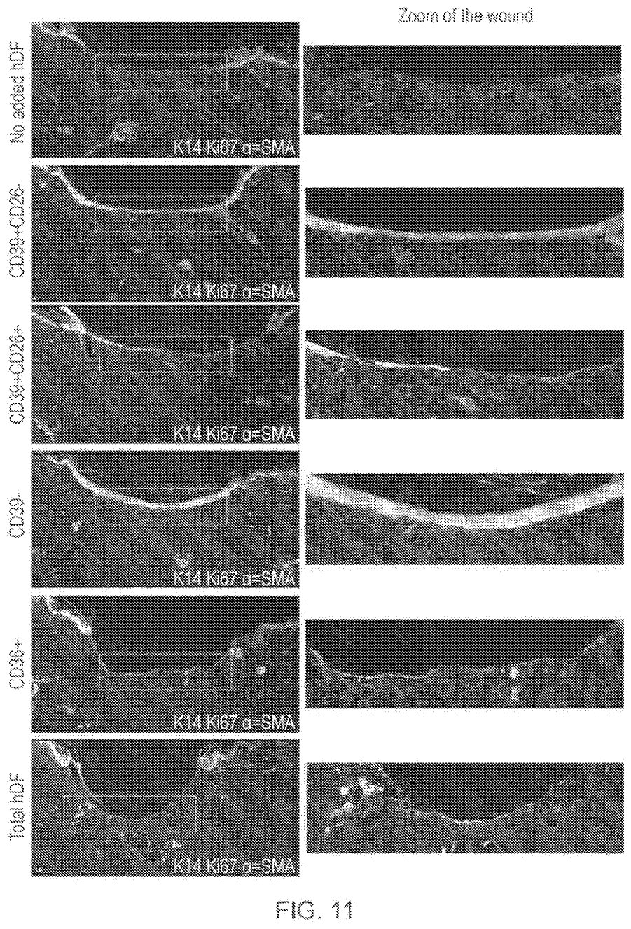

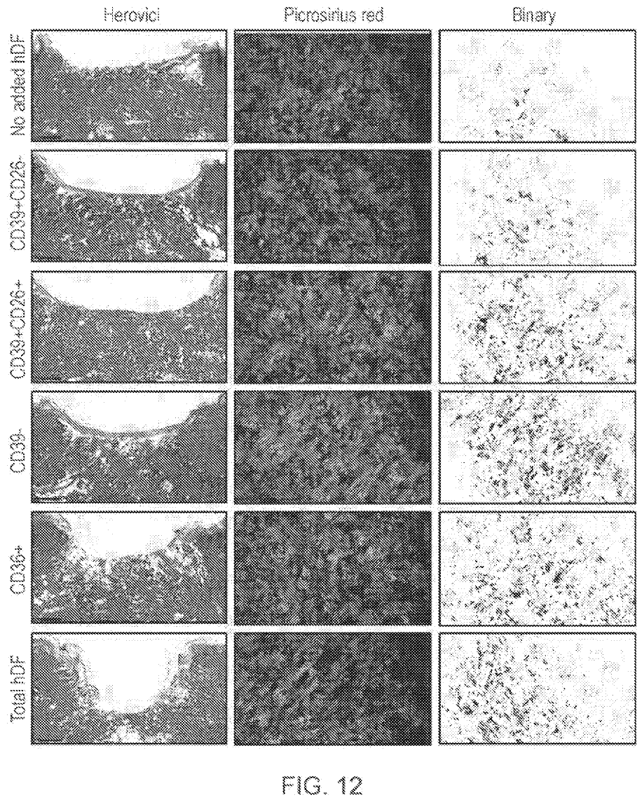

Fibroblast subpopulations have a different effect on ex vivo wound healing (see FIGS. 9 to 12). Only papillary CD39+CD26− and reticular CD39-cells were able to support re-epithelialisation. However, CD39-cells secreted significantly more collagen than any other subpopulation. This may have clinical implications for people more prone to fibrosis. Thus, injecting CD39+CD26-cells into wounds can facilitate faster healing without the risk of scar tissue formation.

Differences in Collagen Deposition in Decellularized Dermis Reconstituted with Different Fibroblast Subpopulations

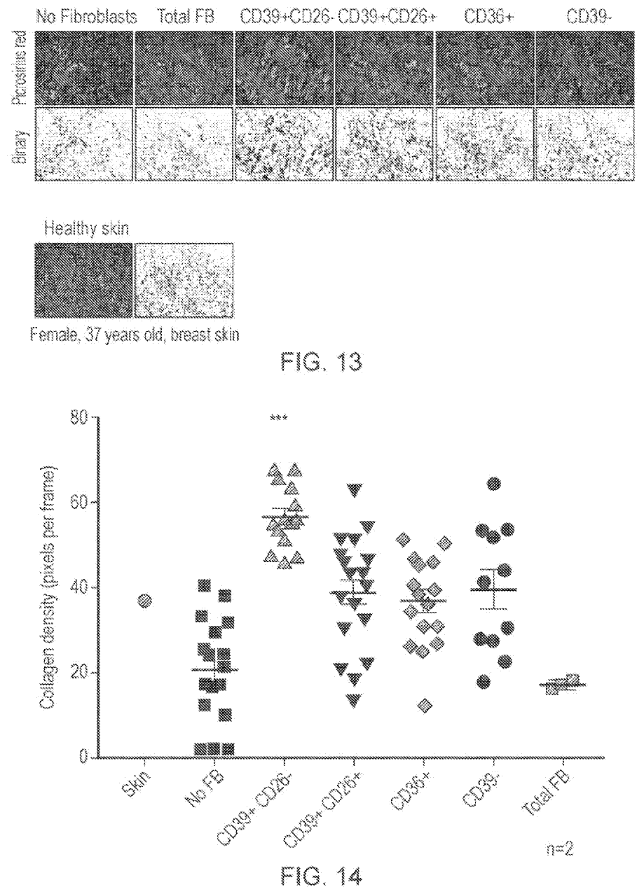

To calculate total collagen density, we stained sections with Picrosirius Red where collagen bundles appear green, red or yellow under polarised light (see FIG. 13). To quantify these images, we converted the total collagen signal into black pixels in a binary image and measured the total number of pixels in the frame. DEDs repopulated with CD39+CD26−papillary fibroblasts have more total collagen density, compared to the other fibroblast subpopulations (see FIG. 14).

Differences in Collagen Fibre Structure in Decellularized Dermis Reconstituted with Different Fibroblast Subpopulations

The collagen hybridizing peptide (CHP) intercalates into accessible collagen triple helixes in maturing and remodelling collagen fibres. It is an extremely specific probe for unfolded collagen molecules and therefore it does not bind to thick, triple helical matured collagen. CD39+CD26−papillary fibroblasts appear to be producing more new collagen fibres and/or remodelling existing collagens in the DEDs, compared to the other fibroblast subpopulations (see FIG. 15).

Epidermal WNT Signalling in Decellularized Dermis Reconstituted with Different Fibroblast Subpopulations

CD39+CD26−papillary fibroblasts stimulate higher levels of R-spondin1 in the epidermis (see FIG. 16).

### Keloid Scar Remodelling by Fibroblast Subpopulations.

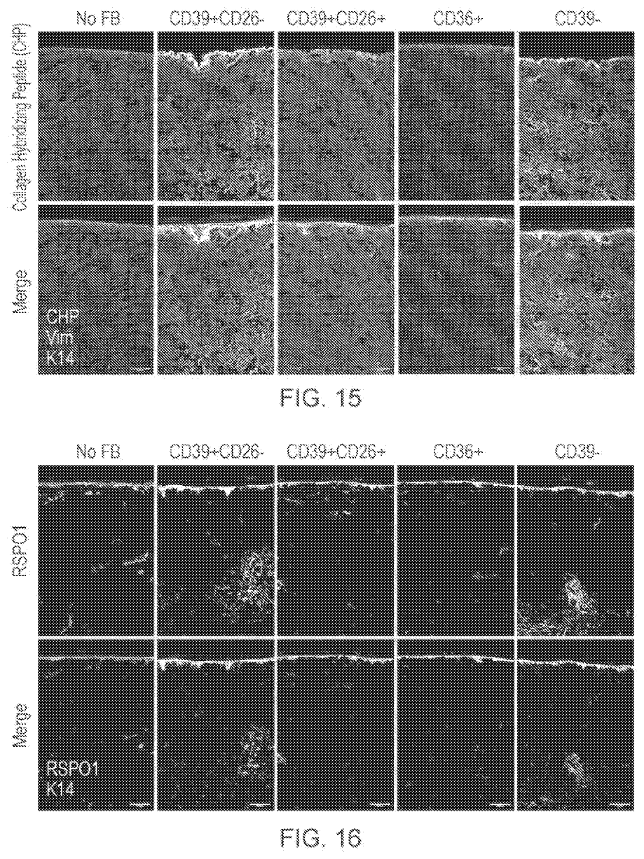

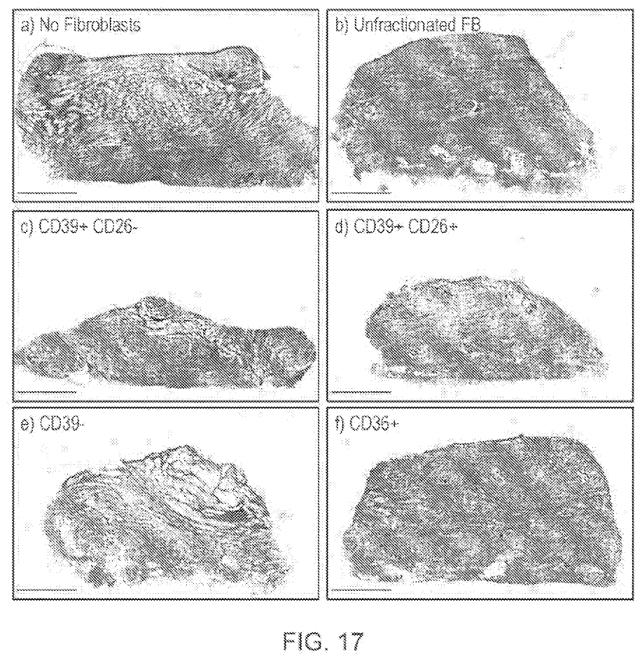

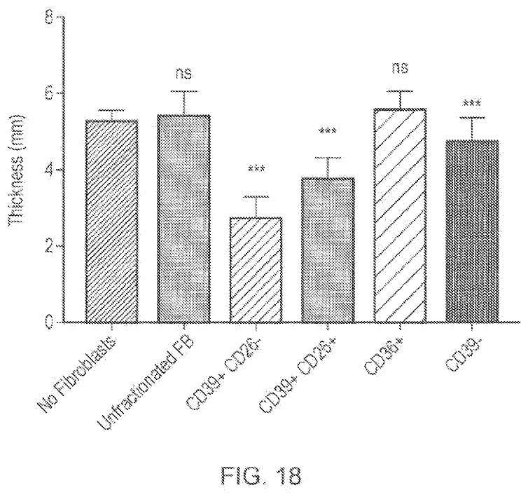

Keloid tissue was decellularized, injected with different fibroblast subpopulations, and cultured in vitro for 3 weeks. Papillary fibroblasts (CD39+CD26−) (c) reduced scar tissue thickness more than other isolated populations (see FIG. 17, b, d, e & f). Unfractionated (b) and CD36+ reticular/pre-adipocyte (f) fibroblasts had no effect on scar remodelling, when compared to the control tissue with no added fibroblasts (see FIG. 18).

### DISCUSSION

The results show differential effects of individual fibroblast subpopulations in wound healing, collagen deposition and scarring. In a wound healing model, papillary (CD39+CD26−) fibroblasts enhanced re-epithelialisation without increasing collagen production. Papillary (CD39+CD26−) fibroblasts also reduced scar tissue thickness more than other populations during keloid tissue remodelling. This suggests the use of this subpopulation to promote wound healing with reduced risk of scarring. In contrast, in decelluarized dermis papillary (CD39+CD26−) fibroblasts promoted higher collagen production compared to other fibroblast subpopulations, implying their use for repairing damaged collagen, for example collagen damage due to exposure to ultraviolet light.

### REFERENCES

1 Kretzschmar, K. & Watt, F. M. Lineage tracing. Cell 148, 33-45, doi:10.1016/j.cell.2012.01.002 (2012).
2 Driskell, R. R. & Watt, F. M. Understanding fibroblast heterogeneity in the skin. Trends in cell biology 25, 92-99, doi: 10.1016/j.tcb.2014.10.001 (2015).
3 Kalluri, R. & Zeisberg, M. Fibroblasts in cancer. Nature reviews. Cancer 6, 392-401, doi:10.1038/nrc1877 (2006).
4 Parsonage, G. et al. A stromal address code defined by fibroblasts. Trends in immunology 26, 150-156, doi:10.1016/j.it.2004.11.014 (2005).
5 Tomasek, J. J., Gabbiani, G., Hinz, B., Chaponnier, C. & Brown, R. A. Myofibroblasts and mechano-regulation of connective tissue remodelling. Nature reviews. Molecular cell biology 3, 349-363, doi: 10.1038/nrm809 (2002).
6 Watt, F. M. & Fujiwara, H. Cell-extracellular matrix interactions in normal and diseased skin. Cold Spring Harbor perspectives in biology 3, doi:10.1101/cshperspect.a005124 (2011).
7 Harper, R. A. & Grove, G. Human skin fibroblasts derived from papillary and reticular dermis: differences in growth potential in vitro. Science 204, 526-527 (1979).
8 Festa, E. et al. Adipocyte lineage cells contribute to the skin stem cell niche to drive hair cycling. Cell 146, 761-771, doi: 10.1016/j.cell.2011.07.019 (2011).
9 Driskell, R. R., Jahoda, C. A., Chuong, C. M., Watt, F. M. & Horsley, V. Defining dermal adipose tissue. Experimental dermatology 23, 629-631, doi: 10.1111/exd. 12450 (2014).
10 Lee, J. & Tumbar, T. Hairy tale of signaling in hair follicle development and cycling. Seminars in cell & developmental biology 23, 906-916, doi:10.1016/j.semcdb.2012.08.003 (2012).
11 Sennett, R. & Rendl, M. Mesenchymal-epithelial interactions during hair follicle morphogenesis and cycling. Seminars in cell & developmental biology 23, 917-927, doi:10.1016/j.semcdb.2012.08.011 (2012).
12 Paquet-Fifield, S. et al. A role for pericytes as microenvironmental regulators of human skin tissue regeneration. The Journal of clinical investigation 119, 2795-2806, doi:10.1172/JCI38535 (2009).
13 Driskell, R. R. et al. Distinct fibroblast lineages determine dermal architecture in skin development and repair. Nature 504, 277-281, doi: 10.1038/nature12783 (2013).
14 Rinkevich, Y. et al. Skin fibrosis. Identification and isolation of a dermal lineage with intrinsic fibrogenic potential. Science 348, aaa2151, doi:10.1126/science.aaa2151 (2015).
15 Sorrell, J. M., Baber, M. A. & Caplan, A. I. Site-matched papillary and reticular human dermal fibroblasts differ in their release of specific growth factors/cytokines and in their interaction with keratinocytes. Journal of cellular physiology 200, 134-145, doi:10.1002/jcp. 10474 (2004).
16 Janson, D. G., Saintigny, G., van Adrichem, A., Mahe, C. & El Ghalbzouri, A. Different gene expression patterns in human papillary and reticular fibroblasts. J Invest Dermatol 132, 2565-2572, doi: 10.1038/jid.2012.192 (2012).
17 Lee, D. Y. & Cho, K. H. The effects of epidermal keratinocytes and dermal fibroblasts on the formation of cutaneous basement membrane in three-dimensional culture systems. Archives for dermatological research=Archiv fur dermatologische Forschung 296, 296-302, doi:10.1007/s00403-004-0529-5 (2005).
18 Rognoni, E. et al. Inhibition of beta-catenin signalling in dermal fibroblasts enhances hair follicle regeneration during wound healing. Development 143, 2522-2535, doi:10.1242/dev.131797 (2016).
19 Natarajan, A., Lemos, D. R. & Rossi, F. M. Fibro/adipogenic progenitors: a double-edged sword in skeletal muscle regeneration. Cell cycle 9, 2045-2046, doi: 10.4161/cc.9.11.11854 (2010).
20 Mastrogiannaki, M. et al. beta-Catenin Stabilization in Skin Fibroblasts Causes Fibrotic Lesions by Preventing Adipocyte Differentiation of the Reticular Dermis. J Invest Dermatol 136, 1130-1142, doi:10.1016/j.jid.2016.01.036 (2016).
21 Rivera-Gonzalez, G. C. et al. Skin Adipocyte Stem Cell Self-Renewal Is Regulated by a PDGFA/AKT-Signaling Axis. Cell stem cell 19, 738-751, doi:10.1016/j.stem.2016.09.002 (2016).
22 Kuniyasu, A. et al. CD36-mediated endocytic uptake of advanced glycation end products (AGE) in mouse 3T3-L1 and human subcutaneous adipocytes. FEBS letters 537, 85-90 (2003).
23 Saalbach, A., Kraft, R., Herrmann, K., Haustein, U. F. & Anderegg, U. The monoclonal antibody AS02 recognizes a protein on human fibroblasts being highly homologous to Thy-1. Archives for dermatological research=Archiv fur dermatologische Forschung 290, 360-366 (1998).
24 Fitzgerald, J., Holden, P. & Hansen, U. The expanded collagen VI family: new chains and new questions. Connective tissue research 54, 345-350, doi: 10.3109/03008207.2013.822865 (2013).
25 Gara, S. K. et al. Three novel collagen VI chains with high homology to the alpha3 chain. The Journal of biological chemistry 283, 10658-10670, doi:10.1074/jbc.M709540200 (2008).
26 Fitzgerald, J., Rich, C., Zhou, F. H. & Hansen, U. Three novel collagen VI chains, alpha4(VI), alpha5(VI), and alpha6(VI). The Journal of biological chemistry 283, 20170-20180, doi:10.1074/jbc.M710139200 (2008).
27 Sabatelli, P. et al. Expression of the collagen VI alpha5 and alpha6 chains in normal human skin and in skin of patients with collagen VI-related myopathies. J Invest Dermatol 131, 99-107, doi:10.1038/jid.2010.284 (2011).
28 Martinelli-Boneschi, F. et al. COL6A5 variants in familial neuropathic chronic itch. Brain: a journal of neurology, doi: 10.1093/brain/aww343 (2017).
29 Collins, C. A., Kretzschmar, K. & Watt, F. M. Reprogramming adult dermis to a neonatal state through epidermal activation of beta-catenin. Development 138, 5189-5199, doi:10.1242/dev.064592 (2011).
30 Lichtenberger, B. M., Mastrogiannaki, M. & Watt, F. M. Epidermal beta-catenin activation remodels the dermis via paracrine signalling to distinct fibroblast lineages. Nature communications 7, 10537, doi: 10.1038/ncomms10537 (2016).
31 Kretzschmar, K., Weber, C., Driskell, R. R., Calonje, E. & Watt, F. M. Compartmentalized Epidermal Activation of beta-Catenin Differentially Affects Lineage Reprogramming and Underlies Tumor Heterogeneity. Cell reports 14, 269-281, doi:10.1016/j.celrep.2015.12.041 (2016).
32 Karakikes, I. et al. Interaction of HLA-DR and CD74 at the cell surface of antigen-presenting cells by single particle image analysis. FASEB journal: official publication of the Federation of American Societies for Experimental Biology 26, 4886-4896, doi: 10.1096/fj.12-211466 (2012).
33 Findley, M. K. & Koval, M. Regulation and roles for claudin-family tight junction proteins. IUBMB life 61, 431-437, doi: 10.1002/iub.175 (2009).
34 Brocks, T. et al. Macrophage migration inhibitory factor protects from nonmelanoma epidermal tumors by regulating the number of antigen-presenting cells in skin. FASEB journal: official publication of the Federation of American Societies for Experimental Biology 31, 526-543, doi: 10.1096/fj.201600860R (2017).
35 Kluger, M. S., Clark, P. R., Tellides, G., Gerke, V. & Pober, J. S. Claudin-5 controls intercellular barriers of human dermal microvascular but not human umbilical vein endothelial cells. Arteriosclerosis, thrombosis, and vascular biology 33, 489-500, doi:10.1161/ATVBAHA.112.300893 (2013).
36 Patel, A. S. et al. TIE2-expressing monocytes/macrophages regulate revascularization of the ischemic limb. EMBO molecular medicine 5, 858-869, doi: 10.1002/emmm.201302752 (2013).
37 Bondjers, C. et al. Transcription profiling of platelet-derived growth factor-B-deficient mouse embryos identifies RGS5 as a novel marker for pericytes and vascular smooth muscle cells. The American journal of pathology 162, 721-729, doi: 10.1016/S0002-9440(10)63868-0 (2003).
38 Rendl, M., Lewis, L. & Fuchs, E. Molecular dissection of mesenchymal-epithelial interactions in the hair follicle. PLOS biology 3, e331, doi: 10.1371/journal.pbio.0030331 (2005)
39 Driskell, R. R., Giangreco, A., Jensen, K. B., Mulder, K. W. & Watt, F. M. Sox2-positive dermal papilla cells specify hair follicle type in mammalian epidermis. Development 136, 2815-2823, doi:10.1242/dev.038620 (2009).
40 Driskell, R. R. et al. Clonal growth of dermal papilla cells in hydrogels reveals intrinsic differences between Sox2-positive and -negative cells in vitro and in vivo. J Invest Dermatol 132, 1084-1093, doi:10.1038/jid.2011.428 (2012).
41 Enshell-Seijffers, D., Lindon, C. & Morgan, B. A. The serine protease Corin is a novel modifier of the Agouti pathway. Development 135, 217-225, doi: 10.1242/dev.011031 (2008).
42 Ng, Y. Y., Baert, M. R., de Haas, E. F., Pike-Overzet, K. & Staal, F. J. Isolation of human and mouse hematopoietic stem cells. Methods in molecular biology 506, 13-21, doi: 10.1007/978-1-59745-409-4_2 (2009).
43 Boonacker, E. & Van Noorden, C. J. The multifunctional or moonlighting protein CD26/DPPIV. European journal of cell biology 82, 53-73, doi: 10.1078/0171-9335-00302 (2003).
44 Hu, M. S. & Longaker, M. T. Dipeptidyl Peptidase-4, Wound Healing, Scarring, and Fibrosis. Plastic and reconstructive surgery 138, 1026-1031, doi:10.1097/PRS.0000000000002634 (2016).
45 Ohtola, J. et al. beta-Catenin has sequential roles in the survival and specification of ventral dermis. Development 135, 2321-2329, doi:10.1242/dev.021170 (2008).
46 Grzeschik, K. H. et al. Deficiency of PORCN, a regulator of Wnt signaling, is associated with focal dermal hypoplasia. Nature genetics 39, 833-835, doi: 10.1038/ng2052 (2007).
47 Kishimoto, J., Burgeson, R. E. & Morgan, B. A. Wnt signaling maintains the hair-inducing activity of the dermal papilla. Genes & development 14, 1181-1185 (2000).
48 Myung, P. S., Takeo, M., Ito, M. & Atit, R. P. Epithelial Wnt ligand secretion is required for adult hair follicle growth and regeneration. J Invest Dermatol 133, 31-41, doi:10.1038/jid.2012.230 (2013).
49 Silva-Vargas, V. et al. Beta-catenin and Hedgehog signal strength can specify number and location of hair follicles in adult epidermis without recruitment of bulge stem cells. Developmental cell 9, 121-131, doi:10.1016/j.devcel.2005.04.013 (2005).
50 Donati, G. et al. Epidermal Wnt/beta-catenin signaling regulates adipocyte differentiation via secretion of adipogenic factors. Proceedings of the National Academy of Sciences of the United States of America 111, E1501-1509, doi:10.1073/pnas. 1312880111 (2014).
51 Watt, F. M., Lo Celso, C. & Silva-Vargas, V. Epidermal stem cells: an update. Current opinion in genetics & development 16, 518-524, doi:10.1016/j.gde.2006.08.006 (2006).
52 Watt, F. M. & Jensen, K. B. Epidermal stem cell diversity and quiescence. EMBO molecular medicine 1, 260-267, doi: 10.1002/emmm.200900033 (2009).
53 Gurtner, G. C., Werner, S., Barrandon, Y. & Longaker, M. T. Wound repair and regeneration. Nature 453, 314-321, doi: 10.1038/nature07039 (2008).
54 Jumper, N., Paus, R. & Bayat, A. Functional histopathology of keloid disease. Histology and histopathology 30, 1033-1057, doi: 10.14670/HH-11-624 (2015).
55 Lee, J. Y., Yang, C. C., Chao, S. C. & Wong, T. W. Histopathological differential diagnosis of keloid and hypertrophic scar. The American Journal of dermatopathology 26, 379-384 (2004).
56 Gilchrest, B. A. A review of skin ageing and its medical therapy. Br J Dermatol 135, 867-875 (1996).
57 El-Domyati, M. et al. Intrinsic aging vs. photoaging: a comparative histopathological, immunohistochemical, and ultrastructural study of skin. Experimental dermatology 11, 398-405 (2002).
58 Dulauroy, S., Di Carlo, S. E., Langa, F., Eberl, G. & Peduto, L. Lineage tracing and genetic ablation of ADAM12(+) perivascular cells identify a major source of profibrotic cells during acute tissue injury. Nature medicine 18, 1262-1270, doi: 10.1038/nm.2848 (2012).
59 Kirsner, R. S. et al. Spray-applied cell therapy with human allogeneic fibroblasts and keratinocytes for the treatment of chronic venous leg ulcers: a phase 2, multicentre, double-blind, randomised, placebo-controlled trial. Lancet 380, 977-985, doi: 10.1016/S0140-6736(12)60644-8 (2012).
60 Wong, T. et al. Potential of fibroblast cell therapy for recessive dystrophic epidermolysis bullosa. J Invest Dermatol 128, 2179-2189, doi: 10.1038/jid.2008.78 (2008).
61 Sorrell, J. M., Baber, M. A. & Caplan, A. I. Human dermal fibroblast subpopulations; differential interactions with vascular endothelial cells in coculture: nonsoluble factors in the extracellular matrix influence interactions. Wound repair and regeneration: official publication of the Wound Healing Society [and] the European Tissue Repair Society 16, 300-309, doi: 10.1111/j.1524-475X.2008.00369.x (2008).
62 Jensen, K. B., Driskell, R. R. & Watt, F. M. Assaying proliferation and differentiation capacity of stem cells using disaggregated adult mouse epidermis. Nature protocols 5, 898-911, doi:10.1038/nprot.2010.39 (2010).
63 Rheinwald, J. G. & Green, H. Serial cultivation of strains of human epidermal keratinocytes: the formation of keratinizing colonies from single cells. Cell 6, 331-343 (1975).
64 Jones, P. H. & Watt, F. M. Separation of human epidermal stem cells from transit amplifying cells on the basis of differences in integrin function and expression. Cell 73, 713-724 (1993).
65 Rikimaru, K., Moles, J. P. & Watt, F. M. Correlation between hyperproliferation and suprabasal integrin expression in human epidermis reconstituted in culture. Experimental dermatology 6, 214-221 (1997).
66 Picelli, S. et al. Full-length RNA-seq from single cells using Smart-seq2. Nature protocols 9, 171-181, doi:10.1038/nprot.2014.006 (2014).
67 Kim, D. et al. TopHat2: accurate alignment of transcriptomes in the presence of insertions, deletions and gene fusions. Genome biology 14, R36, doi: 10.1186/gb-2013-14-4-r36 (2013).
68 Liao, Y., Smyth, G. K. & Shi, W. featureCounts: an efficient general purpose program for assigning sequence reads to genomic features. Bioinformatics 30, 923-930, doi: 10.1093/bioinformatics/btt656 (2014).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1-21. (canceled)

22. The method for preventing or treating a skin disorder, skin ageing or scarring in a human subject, comprising administering an isolated subpopulation of human dermal fibroblasts to skin of the subject, wherein the human dermal fibroblast subpopulation is isolated based on expression of one or more cell-surface markers selected from CD39, CD36 and CD26

23. The method according to claim 22, wherein the human dermal fibroblasts are autologous to the subject.

24. The method according to claim 22, wherein the human dermal fibroblasts are allogeneic to the subject.

25-29. (canceled)

30. The method according to claim 22, wherein the isolated subpopulation of human dermal fibroblasts is characterized by expression of a cell surface phenotype CD45− CD31− CD324− CD90+ CD39+ CD26−.

31. The method according to claim 30, wherein the isolated subpopulation of human dermal fibroblasts are papillary fibroblasts.

32. The method according to claim 22, wherein the isolated subpopulation of human dermal fibroblasts is characterized by expression of a cell surface phenotype CD45− CD31− CD324− CD90+ CD39− or CD45− CD31− CD324− CD90+ CD39+ CD26+.

33. The method according to claim 32, wherein the isolated subpopulation of human dermal fibroblasts are reticular fibroblasts.

34. The method according to claim 22, wherein the isolated subpopulation of human dermal fibroblasts is characterized by expression of a cell surface phenotype CD45− CD31− CD324− CD90+ CD36+.

35. The method according to claim 34, wherein the isolated subpopulation of human dermal fibroblasts are pre-adipocyte fibroblasts,

36. The method according to claim 22, wherein the isolated subpopulation of human dermal fibroblasts is combined with a pharmacologically acceptable excipient, diluent or carrier.

37. The method according to claim 22, wherein the isolated subpopulation of human dermal fibroblasts is administered by intradermal injection into the skin of the subject.

38. The method according to claim 22, wherein the subject is administered a dose of 10,000 to 100 million cells.

39. The method according to claim 22, wherein the fibroblasts are used to promote wound healing in the subject.

40. The method according to claim 22, wherein the fibroblasts are used to treat keloidal or non-keloidal scarring, scleroderma, graft versus host disease, skin ulcers, genetic skin disorders, epidermolysis bullosa, fibrosis, wrinkles, or contour facial deformities.

41. The method according to claim 22, wherein the human dermal fibroblast subpopulation is isolated by:
(a) providing a cell population comprising human dermal fibroblasts; and
(b) separating the human dermal fibroblasts into subpopulations based on expression of one or more cell-surface markers selected from CD39, CD36 and CD26

42. The method according to claim 41, wherein the human dermal fibroblasts are separated by flow cytometry.

43. The method according to claim 41, wherein human dermal fibroblasts are separated from other human dermal cells by selecting cells having a cell surface phenotype CD45− CD31− CD324− CD90+.

44. The method according claim 41, wherein a first subpopulation of human dermal fibroblasts is separated by selecting cells having a cell surface phenotype CD39+ CD26−.

45. The method according to claim 41, wherein a second subpopulation of human dermal fibroblasts is separated by selecting cells having a cell surface phenotype CD39− or CD39+CD26+.

46. The method according to claim 41, wherein a third subpopulation of human dermal fibroblasts is separated by selecting cells expressing CD36.
